(19) United States
(12) Patent Application Publication
(10) Pub. No.: US 2014/0303900 A1
(43) Pub. Date: Oct. 9, 2014

(54) MOTION PROFILE TEMPLATES AND MOVEMENT LANGUAGES FOR WEARABLE DEVICES

(71) Applicants: Hosain Sadequr Rahman, San Francisco, CA (US); Richard Lee Drysdale, San Francisco, CA (US); Michael Edward Smith Luna, San Jose, CA (US); Scott Fullam, Palo Alto, CA (US); Travis Austin Bogard, San Francisco, CA (US); Jeremiah Robison, San Francisco, CA (US); Max Everett Utter, II, San Francisco, CA (US); Thomas Alan Donaldson, Nailsworth (GB)

(72) Inventors: Hosain Sadequr Rahman, San Francisco, CA (US); Richard Lee Drysdale, San Francisco, CA (US); Michael Edward Smith Luna, San Jose, CA (US); Scott Fullam, Palo Alto, CA (US); Travis Austin Bogard, San Francisco, CA (US); Jeremiah Robison, San Francisco, CA (US); Max Everett Utter, II, San Francisco, CA (US); Thomas Alan Donaldson, Nailsworth (GB)

(73) Assignee: AliphCom, San Francisco, CA (US)

(21) Appl. No.: 14/244,759

(22) Filed: Apr. 3, 2014

Related U.S. Application Data
(63) Continuation of application No. 14/194,424, filed on Feb. 28, 2014.
Application No. 14/194,424 is a continuation of application No. 13/491,524, filed on Jun. 7, 2012.
Application No. 13/491,524 is a continuation-in-part of application No. 13/158,372, filed on Jun. 10, 2011.
Application No. 13/158,372 is a continuation-in-part of application No. 13/180,320, filed on Jul. 11, 2011, now Pat. No. 8,793,522.
Application No. 13/180,320 is a continuation-in-part of application No. 13/158,416, filed on Jun. 11, 2011.
Application No. 13/158,416 is a continuation-in-part of application No. 13/158,372, filed on Jun. 10, 2011.
Application No. 13/491,524 is a continuation-in-part of application No. 13/180,000, filed on Jul. 11, 2011.
Application No. 13/180,000 is a continuation-in-part of application No. 13/158,372, filed on Jun. 10, 2011.
Application No. 13/158,372 is a continuation-in-part of application No. 13/158,416, filed on Jun. 11, 2011.
Application No. 13/158,416 is a continuation-in-part of application No. 13/158,372, filed on Jun. 10, 2011.
(60) Provisional application No. 61/495,995, filed on Jun. 11, 2011.
(60) Provisional application No. 61/495,994, filed on Jun. 11, 2011.
(60) Provisional application No. 61/495,997, filed on Jun. 11, 2011.
(60) Provisional application No. 61/495,996, filed on Jun. 11, 2011.
(60) Provisional application No. 61/495,995, filed on Jun. 11, 2011.
(60) Provisional application No. 61/495,994, filed on Jun. 11, 2011.
(60) Provisional application No. 61/495,997, filed on Jun. 11, 2011.
(60) Provisional application No. 61/495,996, filed on Jun. 11, 2011.
(60) Provisional application No. 61/495,997, filed on Jun. 11, 2011.
(60) Provisional application No. 61/495,995, filed on Jun. 11, 2011.
(60) Provisional application No. 61/495,994, filed on Jun. 11, 2011.
(60) Provisional application No. 61/495,996, filed on Jun. 11, 2011.
(60) Provisional application No. 61/507,091, filed on Jul. 12, 2011.

(51) Int. Cl. A61B 5/11

(52) U.S. Cl. CPC A61B 5/11
USPC 702/19

(57) ABSTRACT

Techniques for determining a state of a user using wearable devices are described, including receiving motion-related data from a first sensor and user-related data from a second sensor over a time period, determining a plurality of states of a user over the time period as a function of the motion-related data and the user-related data, determining a trend as a function of the plurality of states of the user, and displaying information associated with the trend at a user interface. The first sensor and the second sensor may be coupled to a wearable device. The user-related data may include galvanic skin response data. The information associated with the trend may be displayed at a web browser.

### CROSS-REFERENCE TO RELATED APPLICATIONS

This application is a continuation of U.S. patent application Ser. No. 14/194,424, filed Feb. 28, 2014, which is a continuation of U.S. patent application Ser. No. 13/491,524, filed Jun. 7, 2012, which is a continuation-in-part of U.S. patent application Ser. No. 13/158,372, filed Jun. 10, 2011; U.S. patent application Ser. No. 13/491,524 also is a continuation-in-part of U.S. patent application Ser. No. 13/180,320, filed Jul. 11, 2011, which is a continuation-in-part of prior U.S. patent application Ser. No. 13/158,416, filed Jun. 11, 2011, which is a continuation-in-part of U.S. patent application Ser. No. 13/158,372, filed Jun. 10, 2011, and which claims the benefit of U.S. Provisional Patent Application No. 61/495,995, filed Jun. 11, 2011, U.S. Provisional Patent Application No. 61/495,994, filed Jun. 11, 2011, U.S. Provisional Patent Application No. 61/495,997, filed Jun. 11, 2011, and U.S. Provisional Patent Application No. 61/495,996, filed Jun. 11, 2011; U.S. patent application Ser. No. 13/491,524 also is a continuation-in-part of U.S. patent application Ser. No. 13/180,000, filed Jul. 11, 2011, which is a continuation-in-part of prior U.S. patent application Ser. No. 13/158,372, filed Jun. 10, 2011, and a continuation-in-part of prior U.S. patent application Ser. No. 13/158,416, filed Jun. 11, 2011, which is a continuation-in-part of U.S. patent application Ser. No. 13/158,372, filed Jun. 10, 2011, and which claims the benefit of U.S. Provisional Patent Application No. 61/495,995, filed Jun. 11, 2011, U.S. Provisional Patent Application No. 61/495,994, filed Jun. 11, 2011, U.S. Provisional Patent Application No. 61/495,997, filed Jun. 11, 2011, and U.S. Provisional Patent Application No. 61/495,996, filed Jun. 11, 2011; and U.S. patent application Ser. No. 13/491,524 claims the benefit of U.S. Provisional Patent Application No. 61/495,997, filed Jun. 11, 2011, U.S. Provisional Patent Application No. 61/495,995, filed Jun. 11, 2011, U.S. Provisional Patent Application No. 61/495,994, filed Jun. 11, 2011, U.S. Provisional Patent Application No. 61/495,996, filed Jun. 11, 2011, and U.S. Provisional Patent Application No. 61/507,091, filed Jul. 12, 2011; all of which is hereby incorporated by reference in its entirety for all purposes.

### FIELD

The present invention relates generally to electrical and electronic hardware, computer software, human-computing interfaces, wired and wireless network communications, data processing and computing devices. More specifically, techniques related to motion profile templates and movement languages for wearable devices are described.

### BACKGROUND

With the advent of greater computing capabilities in smaller personal and/or portable form factors and an increasing number of applications (i.e., computer and Internet software or programs) for different uses, consumers (i.e., users) have access to large amounts of personal data. Information and data are often readily available, but poorly captured using conventional data capture devices. Conventional devices typically lack capabilities that can capture, analyze, communicate, or use data in a contextually-meaningful, comprehensive, and efficient manner. Further, conventional solutions are often limited to specific individual purposes or uses, demanding that users invest in multiple devices in order to perform different activities (e.g., a sports watch for tracking time and distance, a GPS receiver for monitoring a hike or run, a cyclometer for gathering cycling data, and others). Although a wide range of data and information is available, conventional devices and applications fail to provide effective solutions that comprehensively capture data for a given user across numerous disparate activities and allow for easy and effective usability solutions. Various types of human-computing interfaces are available with conventional solutions, but typically require manual intervention that could be disruptive to either an activity or state by requiring extensive user interfacing.

Thus, what is needed is a solution for using or interfacing with data capture devices without the limitations of conventional techniques.

### BRIEF DESCRIPTION OF THE DRAWINGS

Various embodiments or examples (“examples”) are disclosed in the following detailed description and the accompanying drawings:

FIG. 1 illustrates an exemplary data-capable band system;

FIG. 2 illustrates a block diagram of an exemplary data-capable band;

FIG. 3 illustrates sensors for use with an exemplary data-capable band;

FIG. 4 illustrates an application architecture for an exemplary data-capable band;

FIG. 5A illustrates representative data types for use with an exemplary data-capable band;

FIG. 5B illustrates representative data types for use with an exemplary data-capable band in fitness-related activities;

FIG. 5C illustrates representative data types for use with an exemplary data-capable band in sleep management activities;

FIG. 5D illustrates representative data types for use with an exemplary data-capable band in medical-related activities;

FIG. 5E illustrates representative data types for use with an exemplary data-capable band in social media/networking-related activities;

FIGS. 6A to 6F depict a variety of motion signatures as input into a band, such as a data-capable band, according to various embodiments;

FIG. 7A illustrates a perspective view of an exemplary data-capable band;

FIG. 7B illustrates a side view of an exemplary data-capable band;

FIG. 8A illustrates a perspective view of an exemplary data-capable band;

FIG. 8B illustrates a side view of an exemplary data-capable band;

FIG. 9A illustrates a perspective view of an exemplary data-capable band;

FIG. 9B illustrates a side view of an exemplary data-capable band;

FIG. 10 illustrates an exemplary computer system suitable for use with a data-capable band;

FIG. 11 depicts an exemplary inference engine of a band configured to detect an activity and/or a mode based on monitored motion;

FIG. 12 depicts a representative implementation of one or more bands and equivalent devices, as wearable devices, to form unique motion profiles;

FIG. 13 depicts an example of a motion capture manager configured to capture motion and portions therefore;

FIG. 14 depicts an example of a motion analyzer configured to evaluate motion-centric events;

FIG. 15 illustrates an exemplary data-capable band system configured to create and share motion profile templates;

FIG. 16A illustrates an exemplary system for wearable device data security;

FIG. 16B illustrates an exemplary system for media device content management using sensory input;

FIG. 16C illustrates an exemplary system for device control using sensory input;

FIG. 16D illustrates an exemplary system for movement languages in wearable devices;

FIG. 17A illustrates an exemplary process for media device content management using sensory input;

FIG. 17B illustrates an exemplary process for device control using sensory input;

FIG. 17C illustrates an exemplary process for wearable device data security;

FIG. 17D illustrates an exemplary process for movement languages in wearable devices; and

FIG. 18 illustrates an exemplary system for creating, storing, and performing other operations with regard to, motion profile templates.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

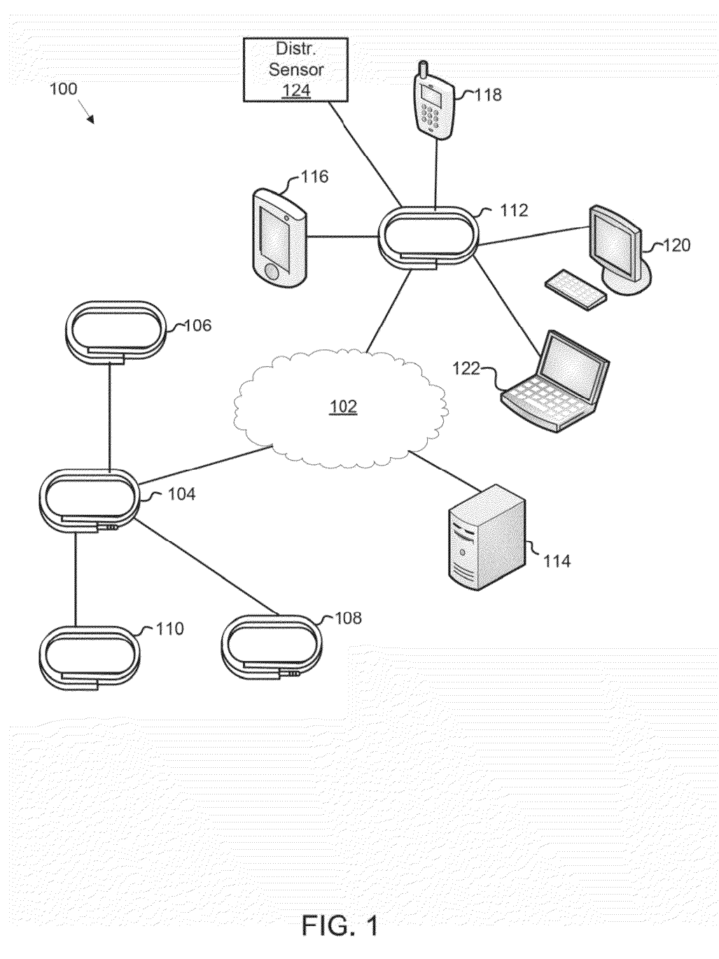

FIG. 1 illustrates an exemplary data-capable band system. Here, system 100 includes network 102, bands 104-112, server 114, mobile computing device 116, mobile communications device 118, computer 120, laptop 122, and distributed sensor 124. Bands 104-112 may be implemented as a data-capable device that may be worn as a strap or band around an arm, leg, ankle, or other bodily appendage or feature. In other examples, bands 104-112 may be attached directly or indirectly to other items, organic or inorganic, animate, or static. In still other examples, bands 104-112 may be used differently.

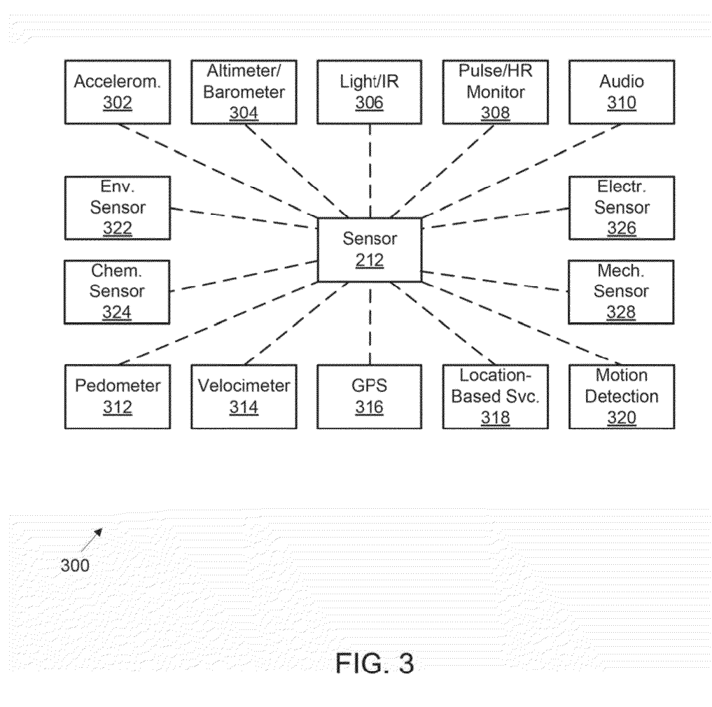

As described above, bands 104-112 may be implemented as wearable personal data or data capture devices (e.g., data-capable devices) that are worn by a user around a wrist, ankle, arm, ear, or other appendage, or attached to the body or affixed to clothing. One or more facilities, sensing elements, or sensors, both active and passive, may be implemented as part of bands 104-112 in order to capture various types of data from different sources. Temperature, environmental, temporal, motion, electronic, electrical, chemical, or other types of sensors (including those described below in connection with FIG. 3) may be used in order to gather varying amounts of data, which may be configurable by a user, locally (e.g., using user interface facilities such as buttons, switches, motion-activated/detected command structures (e.g., accelerometer-gathered data from user-initiated motion of bands 104-112), and others) or remotely (e.g., entering rules or parameters in a website or graphical user interface (“GUI”) that may be used to modify control systems or signals in firmware, circuitry, hardware, and software implemented (i.e., installed) on bands 104-112). Bands 104-112 may also be implemented as data-capable devices that are configured for data communication using various types of communications infrastructure and media, as described in greater detail below. Bands 104-112 may also be wearable, personal, non-intrusive, lightweight devices that are configured to gather large amounts of personally relevant data that can be used to improve user health, fitness levels, medical conditions, athletic performance, sleeping physiology, and physiological conditions, or used as a sensory-based user interface (“UI”) to signal social-related notifications specifying the state of the user through vibration, heat, lights or other sensory based notifications. For example, a social-related notification signal indicating a user is on-line can be transmitted to a recipient, who in turn, receives the notification as, for instance, a vibration.

Using data gathered by bands 104-112, applications may be used to perform various analyses and evaluations that can generate information as to a person's physical (e.g., healthy, sick, weakened, or other states, or activity level), emotional, or mental state (e.g., an elevated body temperature or heart rate may indicate stress, a lowered heart rate and skin temperature, or reduced movement (e.g., excessive sleeping), may indicate physiological depression caused by exertion or other factors, chemical data gathered from evaluating outgassing from the skin's surface may be analyzed to determine whether a person's diet is balanced or if various nutrients are lacking, salinity detectors may be evaluated to determine if high, lower, or proper blood sugar levels are present for diabetes management, and others). Generally, bands 104-112 may be configured to gather from sensors locally and remotely.

As an example, band 104 may capture (i.e., record, store, communicate (i.e., send or receive), process, or the like) data from various sources (i.e., sensors that are organic (i.e., installed, integrated, or otherwise implemented with band 104) or distributed (e.g., microphones on mobile computing device 116, mobile communications device 118, computer 120, laptop 122, distributed sensor 124, global positioning system (“GPS”) satellites, or others, without limitation)) and exchange data with one or more of bands 106-112, server 114, mobile computing device 116, mobile communications device 118, computer 120, laptop 122, and distributed sensor 124. As shown here, a local sensor may be one that is incorporated, integrated, or otherwise implemented with bands 104-112. A remote or distributed sensor (e.g., mobile computing device 116, mobile communications device 118, computer 120, laptop 122, or, generally, distributed sensor 124) may be sensors that can be accessed, controlled, or otherwise used by bands 104-112. For example, band 112 may be configured to control devices that are also controlled by a given user (e.g., mobile computing device 116, mobile communications device 118, computer 120, laptop 122, and distributed sensor 124). For example, a microphone in mobile communications device 118 may be used to detect, for example, ambient audio data that is used to help identify a person's location, or an ear clip (e.g., a headset as described below) affixed to an ear may be used to record pulse or blood oxygen saturation levels. Additionally, a sensor implemented with a screen on mobile computing device 116 may be used to read a user's temperature or obtain a biometric signature while a user is interacting with data. A further example may include using data that is observed on computer 120 or laptop 122 that provides information as to a user's online behavior and the type of content that she is viewing, which may be used by bands 104-112. Regardless of the type or location of sensor used, data may be transferred to bands 104-112 by using, for example, an analog audio jack, digital adapter (e.g., USB, mini-USB), or other, without limitation, plug, or other type of connector that may be used to physically couple bands 104-112 to another device or system for transferring data and, in some examples, to provide power to recharge a battery (not shown). Alternatively, a wireless data communication interface or facility (e.g., a wireless radio that is configured to communicate data from bands 104-112 using one or more data communication protocols (e.g., IEEE 802.11a/b/g/n (WiFi), WiMax, ANT™, ZigBee®, Bluetooth®, Near Field Communications (“NFC”), and others)) may be used to receive or transfer data. Further, bands 104-112 may be configured to analyze, evaluate, modify, or otherwise use data gathered, either directly or indirectly.

In some examples, bands 104-112 may be configured to share data with each other or with an intermediary facility, such as a database, website, web service, or the like, which may be implemented by server 114. In some embodiments, server 114 can be operated by a third party providing, for example, social media-related services (e.g., Facebook®). Bands 104-112 and other related devices may exchange data with each other directly, or bands 104-112 may exchange data via a third party server, such as a third party like Facebook®, to provide social-media related services. Examples of other third party servers include those implemented by social networking services, including, but not limited to, services such as Yahoo! IM™, GTalk™, MSN Messenger™, Twitter® and other private or public social networks. The exchanged data may include personal physiological data and data derived from sensory-based user interfaces (“UI”). Server 114, in some examples, may be implemented using one or more processor-based computing devices or networks, including computing clouds, storage area networks (“SAN”), or the like. As shown, bands 104-112 may be used as a personal data or area network (e.g., “PDN” or “PAN”) in which data relevant to a given user or band (e.g., one or more of bands 104-112) may be shared. As shown here, bands 104 and 112 may be configured to exchange data with each other over network 102 or indirectly using server 114. Users of bands 104 and 112 may direct a web browser hosted on a computer (e.g., computer 120, laptop 122, or the like) in order to access, view, modify, or perform other operations with data captured by bands 104 and 112. For example, two runners using bands 104 and 112 may be geographically remote (e.g., users are not geographically in close proximity locally such that bands being used by each user are in direct data communication), but wish to share data regarding their race times (pre, post, or in-race), personal records (i.e., “PR”), target split times, results, performance characteristics (e.g., target heart rate, target VO₂ max, and others), and other information. If both runners (i.e., bands 104 and 112) are engaged in a race on the same day, data can be gathered for comparative analysis and other uses. Further, data can be shared in substantially real-time (taking into account any latencies incurred by data transfer rates, network topologies, or other data network factors) as well as uploaded after a given activity or event has been performed. In other words, data can be captured by the user as it is worn and configured to transfer data using, for example, a wireless network connection (e.g., a wireless network interface card, wireless local area network (“LAN”) card, cell phone, or the like). Data may also be shared in a temporally asynchronous manner in which a wired data connection (e.g., an analog audio plug (and associated software or firmware) configured to transfer digitally encoded data to encoded audio data that may be transferred between bands 104-112 and a plug configured to receive, encode/decode, and process data exchanged) may be used to transfer data from one or more bands 104-112 to various destinations (e.g., another of bands 104-112, server 114, mobile computing device 116, mobile communications device 118, computer 120, laptop 122, and distributed sensor 124). Bands 104-112 may be implemented with various types of wired and/or wireless communication facilities and are not intended to be limited to any specific technology. For example, data may be transferred from bands 104-112 using an analog audio plug (e.g., TRRS, TRS, or others). In other examples, wireless communication facilities using various types of data communication protocols (e.g., WiFi, Bluetooth®, ZigBee®, ANT™, and others) may be implemented as part of bands 104-112, which may include circuitry, firmware, hardware, radios, antennas, processors, microprocessors, memories, or other electrical, electronic, mechanical, or physical elements configured to enable data communication capabilities of various types and characteristics.

As data-capable devices, bands 104-112 may be configured to collect data from a wide range of sources, including onboard (not shown) and distributed sensors (e.g., server 114, mobile computing device 116, mobile communications device 118, computer 120, laptop 122, and distributed sensor 124) or other bands. Some or all data captured may be personal, sensitive, or confidential and various techniques for providing secure storage and access may be implemented. For example, various types of security protocols and algorithms may be used to encode data stored or accessed by bands 104-112. Examples of security protocols and algorithms include authentication, encryption, encoding, private and public key infrastructure, passwords, checksums, hash codes and hash functions (e.g., SHA, SHA-1, MD-5, and the like), or others may be used to prevent undesired access to data captured by bands 104-112. In other examples, data security for bands 104-112 may be implemented differently.

Bands 104-112 may be used as personal wearable, data capture devices that, when worn, are configured to identify a specific, individual user. By evaluating captured data such as motion data from an accelerometer, biometric data such as heart rate, skin galvanic response, and other biometric data, and using analysis techniques, both long and short-term (e.g., software packages or modules of any type, without limitation), a user may have a unique pattern of behavior or motion and/or biometric responses that can be used as a signature for identification. For example, bands 104-112 may gather data regarding an individual person's gait or other unique biometric, physiological or behavioral characteristics. Using, for example, distributed sensor 124, a biometric signature (e.g., fingerprint, retinal or iris vascular pattern, or others) may be gathered and transmitted to bands 104-112 that, when combined with other data, determines that a given user has been properly identified and, as such, authenticated. When bands 104-112 are worn, a user may be identified and authenticated to enable a variety of other functions such as accessing or modifying data, enabling wired or wireless data transmission facilities (i.e., allowing the transfer of data from bands 104-112), modifying functionality or functions of bands 104-112, authenticating or authorizing financial transactions using stored data and information (e.g., credit card, PIN, card security numbers, and the like), running applications that allow for various operations to be performed (e.g., controlling physical security and access by transmitting a security code to a reader that, when authenticated, unlocks a door by turning off current to an electromagnetic lock, and others), and others. Different functions and operations beyond those described may be performed using bands 104-112, which can act as secure, personal, wearable, data-capable devices. The number, type, function, configuration, specifications, structure, or other features of system 100 and the above-described elements may be varied and are not limited to the examples provided.

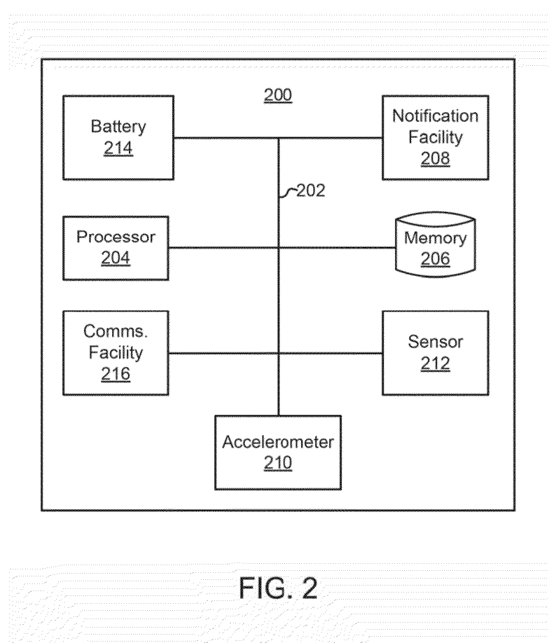

FIG. 2 illustrates a block diagram of an exemplary data-capable band. Here, band 200 includes bus 202, processor 204, memory 206, notification facility 208, accelerometer 210, sensor 212, battery 214, and communications facility 216. In some examples, the quantity, type, function, structure, and configuration of band 200 and the elements (e.g., bus 202, processor 204, memory 206, notification facility 208, accelerometer 210, sensor 212, battery 214, and communications facility 216) shown may be varied and are not limited to the examples provided. As shown, processor 204 may be implemented as logic to provide control functions and signals to memory 206, notification facility 208, accelerometer 210, sensor 212, battery 214, and communications facility 216. Processor 204 may be implemented using any type of processor or microprocessor suitable for packaging within bands 104-112 (FIG. 1). Various types of microprocessors may be used to provide data processing capabilities for band 200 and are not limited to any specific type or capability. For example, a MSP430F5528-type microprocessor manufactured by Texas Instruments of Dallas, Tex. may be configured for data communication using audio tones and enabling the use of an audio plug-and-jack system (e.g., TRRS, TRS, or others) for transferring data captured by band 200. Further, different processors may be desired if other functionality (e.g., the type and number of sensors (e.g., sensor 212)) are varied. Data processed by processor 204 may be stored using, for example, memory 206.

In some examples, memory 206 may be implemented using various types of data storage technologies and standards, including, without limitation, read-only memory (“ROM”), random access memory (“RAM”), dynamic random access memory (“DRAM”), static random access memory (“SRAM”), static/dynamic random access memory (“SDRAM”), magnetic random access memory (“MRAM”), solid state, two and three-dimensional memories, Flash®, and others. Memory 206 may also be implemented using one or more partitions that are configured for multiple types of data storage technologies to allow for non-modifiable (i.e., by a user) software to be installed (e.g., firmware installed on ROM) while also providing for storage of captured data and applications using, for example, RAM. Once captured and/or stored in memory 206, data may be subjected to various operations performed by other elements of band 200.

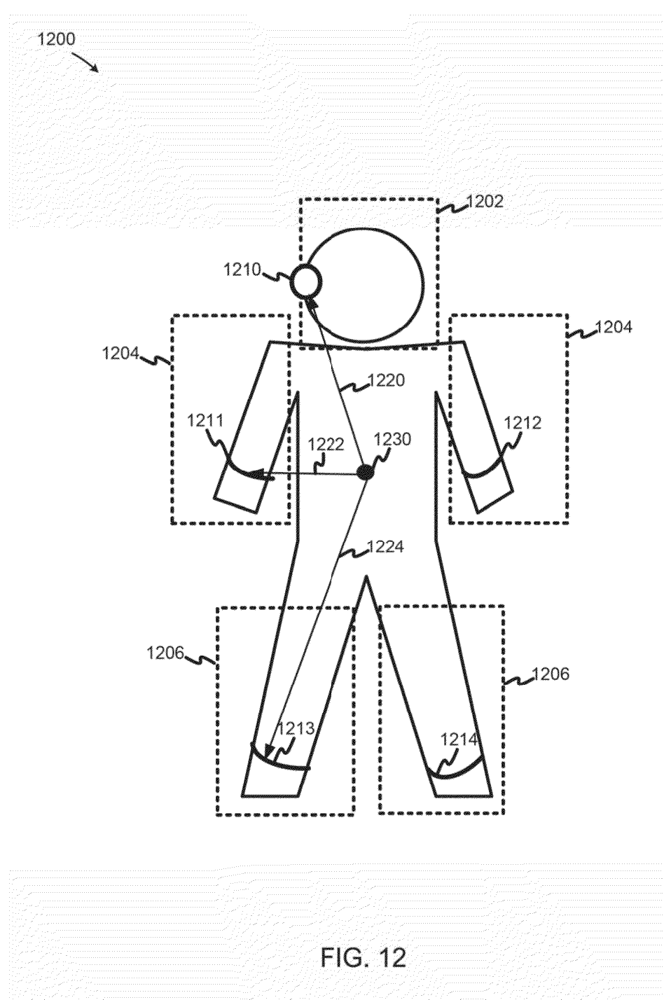

Notification facility 208, in some examples, may be implemented to provide vibratory energy, audio or visual signals, communicated through band 200. As used herein, “facility” refers to any, some, or all of the features and structures that are used to implement a given set of functions. In some examples, the vibratory energy may be implemented using a motor or other mechanical structure. In some examples, the audio signal may be a tone or other audio cue, or it may be implemented using different sounds for different purposes. The audio signals may be emitted directly using notification facility 208, or indirectly by transmission via communications facility 216 to other audio-capable devices (e.g., headphones (not shown), a headset (as described below with regard to FIG. 12), mobile computing device 116, mobile communications device 118, computer 120, laptop 122, distributed sensor 124, etc.). In some examples, the visual signal may be implemented using any available display technology, such as lights, light-emitting diodes (LEDs), interferometric modulator display (IMOD), electrophoretic ink (E Ink), organic light-emitting diode (OLED), or other display technologies. As an example, an application stored on memory 206 may be configured to monitor a clock signal from processor 204 in order to provide timekeeping functions to band 200. For example, if an alarm is set for a desired time, notification facility 208 may be used to provide a vibration or an audio tone, or a series of vibrations or audio tones, when the desired time occurs. As another example, notification facility 208 may be coupled to a framework (not shown) or other structure that is used to translate or communicate vibratory energy throughout the physical structure of band 200. In other examples, notification facility 208 may be implemented differently.

Power may be stored in battery 214, which may be implemented as a battery, battery module, power management module, or the like. Power may also be gathered from local power sources such as solar panels, thermo-electric generators, and kinetic energy generators, among others that are alternatives power sources to external power for a battery. These additional sources can either power the system directly or can charge a battery, which, in turn, is used to power the system (e.g., of a band). In other words, battery 214 may include a rechargeable, expendable, replaceable, or other type of battery, but also circuitry, hardware, or software that may be used in connection with in lieu of processor 204 in order to provide power management, charge/recharging, sleep, or other functions. Further, battery 214 may be implemented using various types of battery technologies, including Lithium Ion (“LI”), Nickel Metal Hydride (“NiMH”), or others, without limitation. Power drawn as electrical current may be distributed from battery via bus 202, the latter of which may be implemented as deposited or formed circuitry or using other forms of circuits or cabling, including flexible circuitry. Electrical current distributed from battery 204 and managed by processor 204 may be used by one or more of memory 206, notification facility 208, accelerometer 210, sensor 212, or communications facility 216.

As shown, various sensors may be used as input sources for data captured by band 200. For example, accelerometer 210 may be used to gather data measured across one, two, or three axes of motion. In addition to accelerometer 210, other sensors (i.e., sensor 212) may be implemented to provide temperature, environmental, physical, chemical, electrical, or other types of sensed inputs. As presented here, sensor 212 may include one or multiple sensors and is not intended to be limiting as to the quantity or type of sensor implemented. Data captured by band 200 using accelerometer 210 and sensor 212 or data requested from another source (i.e., outside of band 200) may also be exchanged, transferred, or otherwise communicated using communications facility 216. For example, communications facility 216 may include a wireless radio, control circuit or logic, antenna, transceiver, receiver, transmitter, resistors, diodes, transistors, or other elements that are used to transmit and receive data from band 200. In some examples, communications facility 216 may be implemented to provide a “wired” data communication capability such as an analog or digital attachment, plug, jack, or the like to allow for data to be transferred. In other examples, communications facility 216 may be implemented to provide a wireless data communication capability to transmit digitally encoded data across one or more frequencies using various types of data communication protocols, without limitation. In still other examples, band 200 and the above-described elements may be varied in function, structure, configuration, or implementation and are not limited to those shown and described.

FIG. 3 illustrates sensors for use with an exemplary data-capable band. Sensor 212 may be implemented using various types of sensors, some of which are shown. Like-numbered and named elements may describe the same or substantially similar element as those shown in other descriptions. Here, sensor 212 (FIG. 2) may be implemented as accelerometer 302, altimeter/barometer 304, light/infrared (“IR”) sensor 306, pulse/heart rate (“HR”) monitor 308, audio sensor (e.g., microphone, transducer, or others) 310, pedometer 312, velocimeter 314, GPS receiver 316, location-based service sensor (e.g., sensor for determining location within a cellular or micro-cellular network, which may or may not use GPS or other satellite constellations for fixing a position) 318, motion detection sensor 320, environmental sensor 322, chemical sensor 324, electrical sensor 326, or mechanical sensor 328.

As shown, accelerometer 302 may be used to capture data associated with motion detection along 1, 2, or 3-axes of measurement, without limitation to any specific type of specification of sensor. Accelerometer 302 may also be implemented to measure various types of user motion and may be configured based on the type of sensor, firmware, software, hardware, or circuitry used. As another example, altimeter/barometer 304 may be used to measure environment pressure, atmospheric or otherwise, and is not limited to any specification or type of pressure-reading device. In some examples, altimeter/barometer 304 may be an altimeter, a barometer, or a combination thereof. For example, altimeter/barometer 304 may be implemented as an altimeter for measuring above ground level (“AGL”) pressure in band 200, which has been configured for use by naval or military aviators. As another example, altimeter/barometer 304 may be implemented as a barometer for reading atmospheric pressure for marine-based applications. In other examples, altimeter/barometer 304 may be implemented differently.

Other types of sensors that may be used to measure light or photonic conditions include light/IR sensor 306, motion detection sensor 320, and environmental sensor 322, the latter of which may include any type of sensor for capturing data associated with environmental conditions beyond light. Further, motion detection sensor 320 may be configured to detect motion using a variety of techniques and technologies, including, but not limited to comparative or differential light analysis (e.g., comparing foreground and background lighting), sound monitoring, or others. Audio sensor 310 may be implemented using any type of device configured to record or capture sound.

In some examples, pedometer 312 may be implemented using devices to measure various types of data associated with pedestrian-oriented activities such as running or walking. Footstrikes, stride length, stride length or interval, time, and other data may be measured. Velocimeter 314 may be implemented, in some examples, to measure velocity (e.g., speed and directional vectors) without limitation to any particular activity. Further, additional sensors that may be used as sensor 212 include those configured to identify or obtain location-based data. For example, GPS receiver 316 may be used to obtain coordinates of the geographic location of band 200 using, for example, various types of signals transmitted by civilian and/or military satellite constellations in low, medium, or high earth orbit (e.g., “LEO,” “MEO,” or “GEO”). In other examples, differential GPS algorithms may also be implemented with GPS receiver 316, which may be used to generate more precise or accurate coordinates. Still further, location-based services sensor 318 may be implemented to obtain location-based data including, but not limited to location, nearby services or items of interest, and the like. As an example, location-based services sensor 318 may be configured to detect an electronic signal, encoded or otherwise, that provides information regarding a physical locale as band 200 passes. The electronic signal may include, in some examples, encoded data regarding the location and information associated therewith. Electrical sensor 326 and mechanical sensor 328 may be configured to include other types (e.g., haptic, kinetic, piezoelectric, piezomechanical, pressure, touch, thermal, and others) of sensors for data input to band 200, without limitation. Other types of sensors apart from those shown may also be used, including magnetic flux sensors such as solid-state compasses and the like, including gyroscopic sensors. While the present illustration provides numerous examples of types of sensors that may be used with band 200 (FIG. 2), others not shown or described may be implemented with or as a substitute for any sensor shown or described.

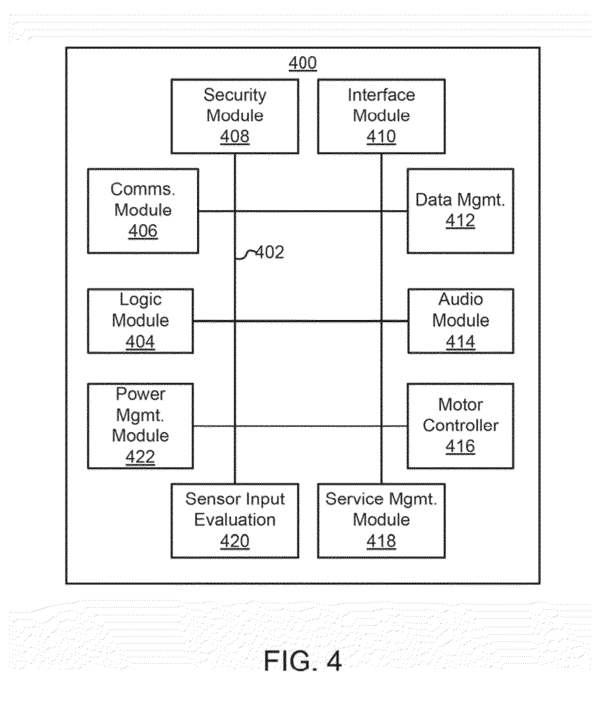

FIG. 4 illustrates an application architecture for an exemplary data-capable band. Here, application architecture 400 includes bus 402, logic module 404, communications module 406, security module 408, interface module 410, data management 412, audio module 414, motor controller 416, service management module 418, sensor input evaluation module 420, and power management module 422. In some examples, application architecture 400 and the above-listed elements (e.g., bus 402, logic module 404, communications module 406, security module 408, interface module 410, data management 412, audio module 414, motor controller 416, service management module 418, sensor input evaluation module 420, and power management module 422) may be implemented as software using various computer programming and formatting languages such as Java, C++, C, and others. As shown here, logic module 404 may be firmware or application software that is installed in memory 206 (FIG. 2) and executed by processor 204 (FIG. 2). Included with logic module 404 may be program instructions or code (e.g., source, object, binary executables, or others) that, when initiated, called, or instantiated, perform various functions.

For example, logic module 404 may be configured to send control signals to communications module 406 in order to transfer, transmit, or receive data stored in memory 206, the latter of which may be managed by a database management system (“DBMS”) or utility in data management module 412. As another example, security module 408 may be controlled by logic module 404 to provide encoding, decoding, encryption, authentication, or other functions to band 200 (FIG. 2). Alternatively, security module 408 may also be implemented as an application that, using data captured from various sensors and stored in memory 206 (and accessed by data management module 412) may be used to provide identification functions that enable band 200 to passively identify a user or wearer of band 200. Still further, various types of security software and applications may be used and are not limited to those shown and described.

Interface module 410, in some examples, may be used to manage user interface controls such as switches, buttons, or other types of controls that enable a user to manage various functions of band 200. For example, a 4-position switch may be turned to a given position that is interpreted by interface module 410 to determine the proper signal or feedback to send to logic module 404 in order to generate a particular result. In other examples, a button (not shown) may be depressed that allows a user to trigger or initiate certain actions by sending another signal to logic module 404. Still further, interface module 410 may be used to interpret data from, for example, accelerometer 210 (FIG. 2) to identify specific movement or motion that initiates or triggers a given response. In other examples, interface module 410 may be used to manage different types of displays (e.g., LED, IMOD, E Ink, OLED, etc.). In other examples, interface module 410 may be implemented differently in function, structure, or configuration and is not limited to those shown and described.

As shown, audio module 414 may be configured to manage encoded or unencoded data gathered from various types of audio sensors. In some examples, audio module 414 may include one or more codecs that are used to encode or decode various types of audio waveforms. For example, analog audio input may be encoded by audio module 414 and, once encoded, sent as a signal or collection of data packets, messages, segments, frames, or the like to logic module 404 for transmission via communications module 406. In other examples, audio module 414 may be implemented differently in function, structure, configuration, or implementation and is not limited to those shown and described. Other elements that may be used by band 200 include motor controller 416, which may be firmware or an application to control a motor or other vibratory energy source (e.g., notification facility 208 (FIG. 2)). Power used for band 200 may be drawn from battery 214 (FIG. 2) and managed by power management module 422, which may be firmware or an application used to manage, with or without user input, how power is consumer, conserved, or otherwise used by band 200 and the above-described elements, including one or more sensors (e.g., sensor 212 (FIG. 2), sensors 302-328 (FIG. 3)). With regard to data captured, sensor input evaluation module 420 may be a software engine or module that is used to evaluate and analyze data received from one or more inputs (e.g., sensors 302-328) to band 200. When received, data may be analyzed by sensor input evaluation module 420, which may include custom or “off-the-shelf” analytics packages that are configured to provide application-specific analysis of data to determine trends, patterns, and other useful information. In other examples, sensor input module 420 may also include firmware or software that enables the generation of various types and formats of reports for presenting data and any analysis performed thereupon.

Another element of application architecture 400 that may be included is service management module 418. In some examples, service management module 418 may be firmware, software, or an application that is configured to manage various aspects and operations associated with executing software-related instructions for band 200. For example, libraries or classes that are used by software or applications on band 200 may be served from an online or networked source. Service management module 418 may be implemented to manage how and when these services are invoked in order to ensure that desired applications are executed properly within application architecture 400. As discrete sets, collections, or groupings of functions, services used by band 200 for various purposes ranging from communications to operating systems to call or document libraries may be managed by service management module 418. Alternatively, service management module 418 may be implemented differently and is not limited to the examples provided herein. Further, application architecture 400 is an example of a software/system/application-level architecture that may be used to implement various software-related aspects of band 200 and may be varied in the quantity, type, configuration, function, structure, or type of programming or formatting languages used, without limitation to any given example.

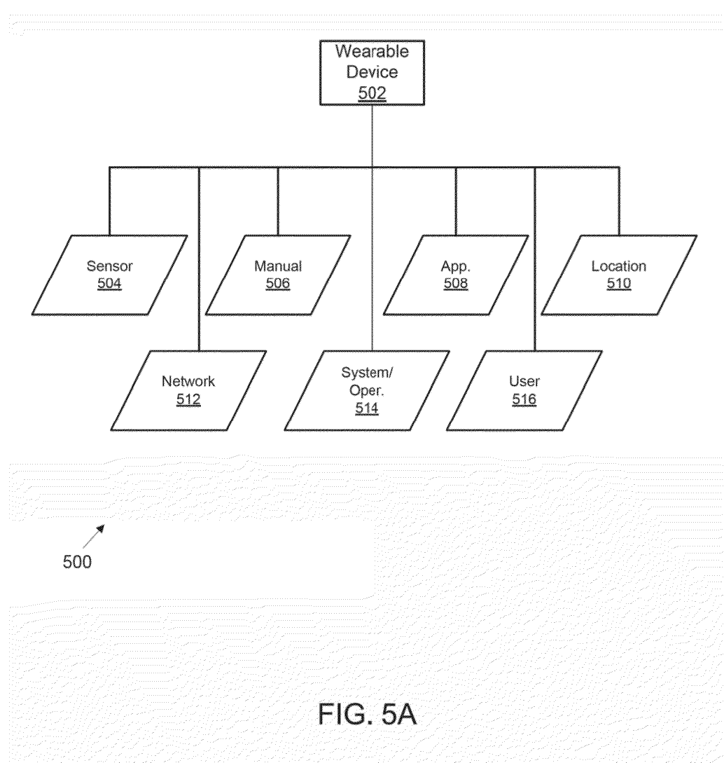

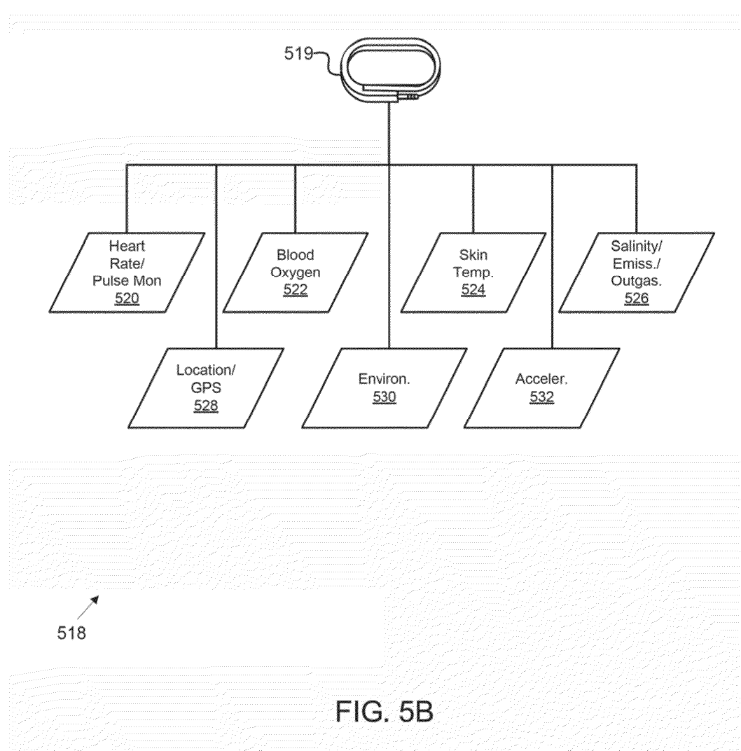

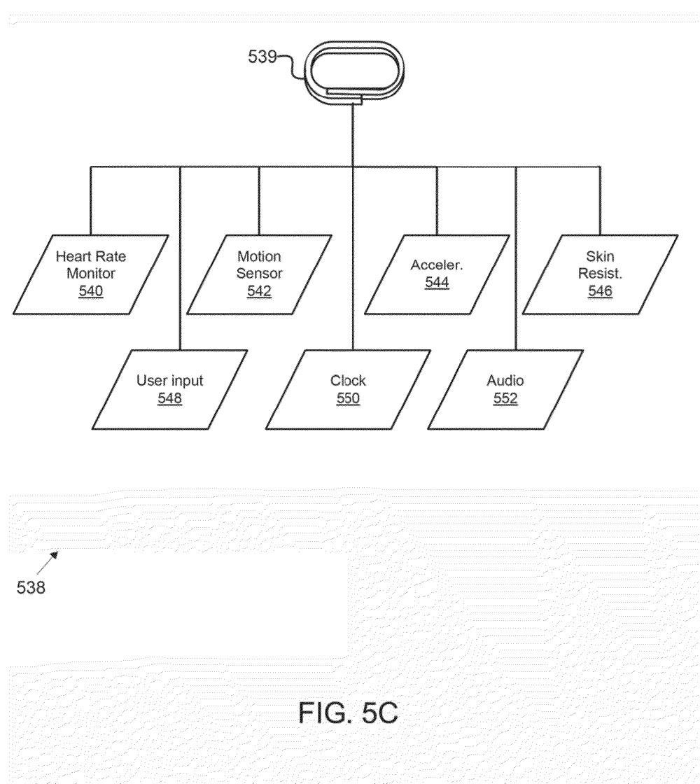

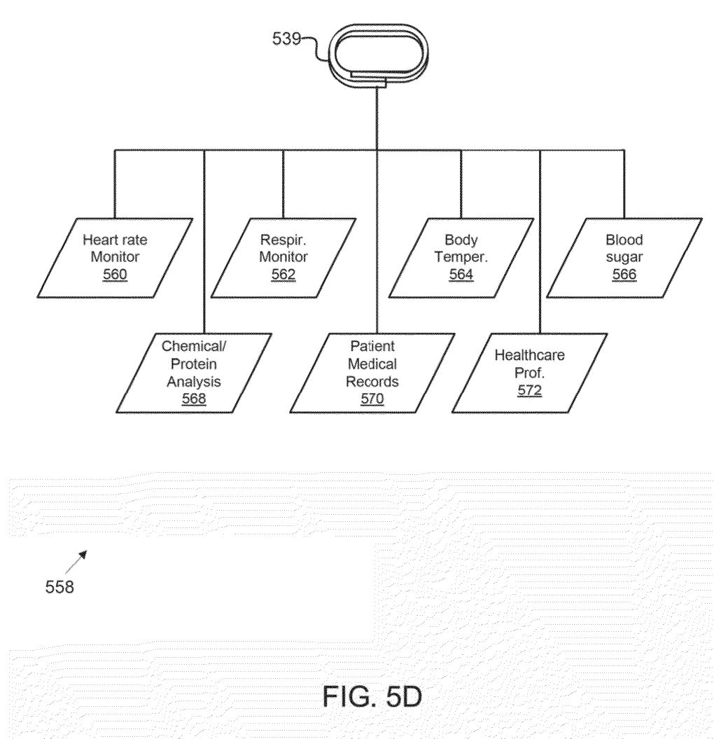

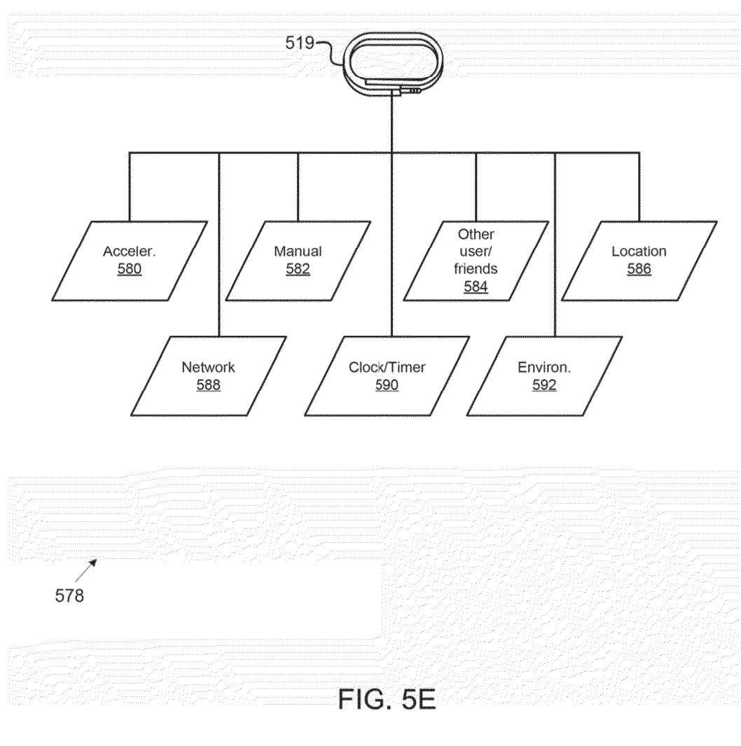

FIG. 5A illustrates representative data types for use with an exemplary data-capable band. Here, wearable device 502 may capture various types of data, including, but not limited to sensor data 504, manually-entered data 506, application data 508, location data 510, network data 512, system/operating data 514, and user data 516. Various types of data may be captured from sensors, such as those described above in connection with FIG. 3. Manually-entered data, in some examples, may be data or inputs received directly and locally by band 200 (FIG. 2). In other examples, manually-entered data may also be provided through a third-party website that stores the data in a database and may be synchronized from server 114 (FIG. 1) with one or more of bands 104-112. Other types of data that may be captured including application data 508 and system/operating data 514, which may be associated with firmware, software, or hardware installed or implemented on band 200. Further, location data 510 may be used by wearable device 502, as described above. User data 516, in some examples, may be data that include profile data, preferences, rules, or other information that has been previously entered by a given user of wearable device 502. Further, network data 512 may be data is captured by wearable device with regard to routing tables, data paths, network or access availability (e.g., wireless network access availability), and the like. Other types of data may be captured by wearable device 502 and are not limited to the examples shown and described. Additional context-specific examples of types of data captured by bands 104-112 (FIG. 1) are provided below.

FIG. 5B illustrates representative data types for use with an exemplary data-capable band in fitness-related activities. Here, band 519 may be configured to capture types (i.e., categories) of data such as heart rate/pulse monitoring data 520, blood oxygen saturation data 522, skin temperature data 524, salinity/emission/outgassing data 526, location/GPS data 528, environmental data 530, and accelerometer data 532. As an example, a runner may use or wear band 519 to obtain data associated with his physiological condition (i.e., heart rate/pulse monitoring data 520, skin temperature, salinity/emission/outgassing data 526, among others), athletic efficiency (i.e., blood oxygen saturation data 522), and performance (i.e., location/GPS data 528 (e.g., distance or laps run), environmental data 530 (e.g., ambient temperature, humidity, pressure, and the like), accelerometer 532 (e.g., biomechanical information, including gait, stride, stride length, among others)). Other or different types of data may be captured by band 519, but the above-described examples are illustrative of some types of data that may be captured by band 519. Further, data captured may be uploaded to a website or online/networked destination for storage and other uses. For example, fitness-related data may be used by applications that are downloaded from a “fitness marketplace” where athletes may find, purchase, or download applications for various uses. Some applications may be activity-specific and thus may be used to modify or alter the data capture capabilities of band 519 accordingly. For example, a fitness marketplace may be a website accessible by various types of mobile and non-mobile clients to locate applications for different exercise or fitness categories such as running, swimming, tennis, golf, baseball, football, fencing, and many others. When downloaded, a fitness marketplace may also be used with user-specific accounts to manage the retrieved applications as well as usage with band 519, or to use the data to provide services such as online personal coaching or targeted advertisements. More, fewer, or different types of data may be captured for fitness-related activities.

FIG. 5C illustrates representative data types for use with an exemplary data-capable band in sleep management activities. Here, band 539 may be used for sleep management purposes to track various types of data, including heart rate monitoring data 540, motion sensor data 542, accelerometer data 544, skin resistivity data 546, user input data 548, clock data 550, and audio data 552. In some examples, heart rate monitor data 540 may be captured to evaluate rest, waking, or various states of sleep. Motion sensor data 542 and accelerometer data 544 may be used to determine whether a user of band 539 is experiencing a restful or fitful sleep. For example, some motion sensor data 542 may be captured by a light sensor that measures ambient or differential light patterns in order to determine whether a user is sleeping on her front, side, or back. Accelerometer data 544 may also be captured to determine whether a user is experiencing gentle or violent disruptions when sleeping, such as those often found in afflictions of sleep apnea or other sleep disorders. Further, skin resistivity data 546 may be captured to determine whether a user is ill (e.g., running a temperature, sweating, experiencing chills, clammy skin, and others). Still further, user input data may include data input by a user as to how and whether band 539 should trigger notification facility 208 (FIG. 2) to wake a user at a given time or whether to use a series of increasing or decreasing vibrations or audio tones to trigger a waking state. Clock data (550) may be used to measure the duration of sleep or a finite period of time in which a user is at rest. Audio data may also be captured to determine whether a user is snoring and, if so, the frequencies and amplitude therein may suggest physical conditions that a user may be interested in knowing (e.g., snoring, breathing interruptions, talking in one's sleep, and the like). More, fewer, or different types of data may be captured for sleep management-related activities.

FIG. 5D illustrates representative data types for use with an exemplary data-capable band in medical-related activities. Here, band 539 may also be configured for medical purposes and related-types of data such as heart rate monitoring data 560, respiratory monitoring data 562, body temperature data 564, blood sugar data 566, chemical protein/analysis data 568, patient medical records data 570, and healthcare professional (e.g., doctor, physician, registered nurse, physician's assistant, dentist, orthopedist, surgeon, and others) data 572. In some examples, data may be captured by band 539 directly from wear by a user. For example, band 539 may be able to sample and analyze sweat through a salinity or moisture detector to identify whether any particular chemicals, proteins, hormones, or other organic or inorganic compounds are present, which can be analyzed by band 539 or communicated to server 114 to perform further analysis. If sent to server 114, further analyses may be performed by a hospital or other medical facility using data captured by band 539. In other examples, more, fewer, or different types of data may be captured for medical-related activities.

FIG. 5E illustrates representative data types for use with an exemplary data-capable band in social media/networking-related activities. Examples of social media/networking-related activities include activities related to Internet-based Social Networking Services (“SNS”), such as Facebook®, Twitter®, etc. Here, band 519, shown with an audio data plug, may be configured to capture data for use with various types of social media and networking-related services, websites, and activities. Accelerometer data 580, manual data 582, other user/friends data 584, location data 586, network data 588, clock/timer data 590, and environmental data 592 are examples of data that may be gathered and shared by, for example, uploading data from band 519 using, for example, an audio plug such as those described herein. As another example, accelerometer data 580 may be captured and shared with other users to share motion, activity, or other movement-oriented data. Manual data 582 may be data that a given user also wishes to share with other users. Likewise, other user/friends data 584 may be from other bands (not shown) that can be shared or aggregated with data captured by band 519. Location data 586 for band 519 may also be shared with other users. In other examples, a user may also enter manual data 582 to prevent other users or friends from receiving updated location data from band 519. Additionally, network data 588 and clock/timer data may be captured and shared with other users to indicate, for example, activities or events that a given user (i.e., wearing band 519) was engaged at certain locations. Further, if a user of band 519 has friends who are not geographically located in close or near proximity (e.g., the user of band 519 is located in San Francisco and her friend is located in Rome), environmental data can be captured by band 519 (e.g., weather, temperature, humidity, sunny or overcast (as interpreted from data captured by a light sensor and combined with captured data for humidity and temperature), among others). In other examples, more, fewer, or different types of data may be captured for medical-related activities.

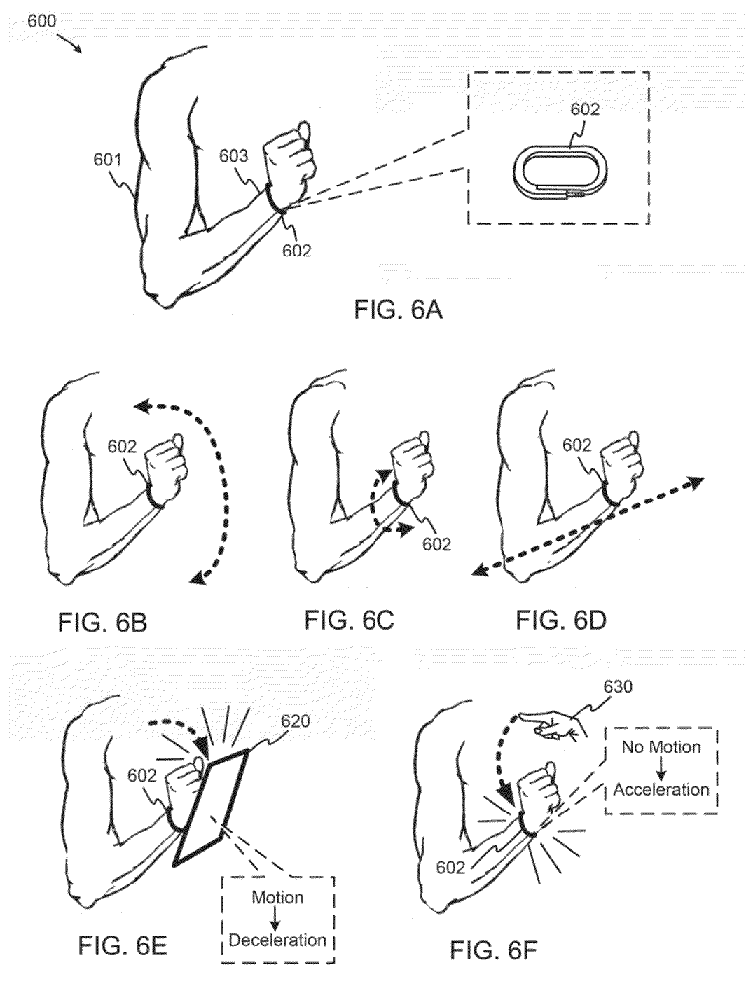

FIG. 6A to 6F depict a variety of motion signatures as input into a band, such as a data-capable band. In FIG. 6A, diagram 600 depicts a user's arm (e.g., as a locomotive member or appendage) with a band 602 attached to user wrist 603. Band 602 can envelop or substantially surround user wrist 603 as well. FIGS. 6B to 6D illustrate different “motion signatures” defined by various ranges of motion and/or motion patterns (as well as number of motions). In some examples, each of the motion signatures may identify a mode of operation. In other examples, a motion signature may provide a different kind of input. For example, FIG. 6B depicts an up-and-down motion, FIG. 6C depicts a rotation about the wrist, and FIG. 6D depicts a side-to-side motion. In another example, FIG. 6E depicts an ability to detect a change in mode as a function of motion and deceleration (e.g., when a user claps hands or makes contact with a surface 620 to get band 602 to change modes). In still another example, FIG. 6F depicts an ability to detect “no motion” initially and experience an abrupt acceleration of the band (e.g., user taps band with finger 630 to change modes). In some examples, motion signatures may be motion patterns that are predetermined, with the user selecting or linking a specific motion signature to invoke a specific mode. In other examples, a user may define unique motion signatures. In some embodiments, any number of detect motions can be used to define a motion signature. Thus, in some examples, different numbers of the same motion can activate different modes. For example, two of the up-and-down motions depicted in FIG. 6B can activate one mode, whereas four up-and-down motions can activate another mode. In other examples, any combination of motions (e.g., two up-and-down motions of FIG. 6B and two taps of FIG. 6E) can be used as an input, regardless of whether a mode of operation or otherwise (e.g., to communicate to another device, to display information, or other action).

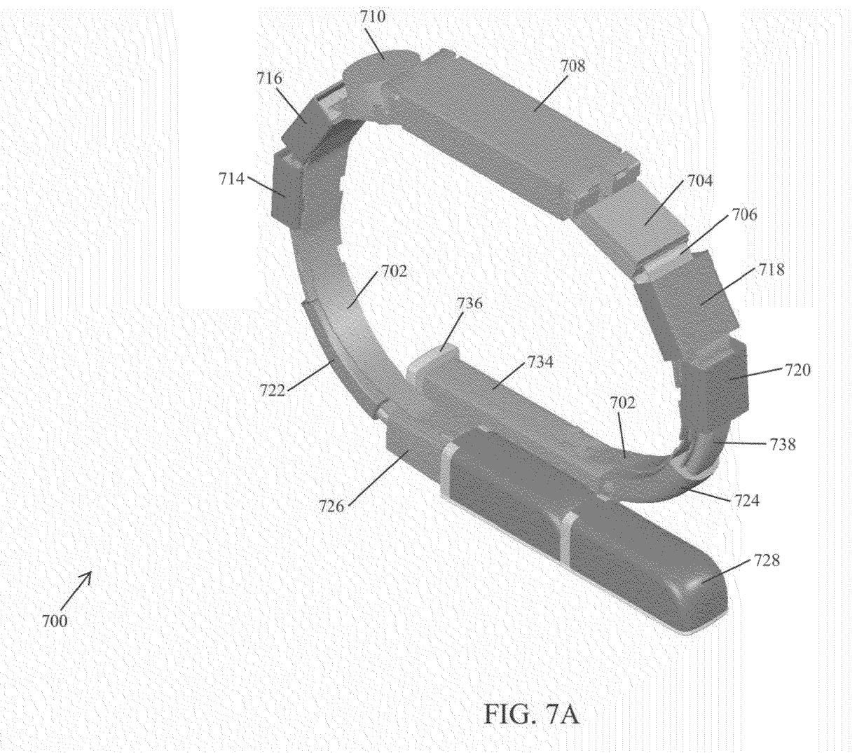

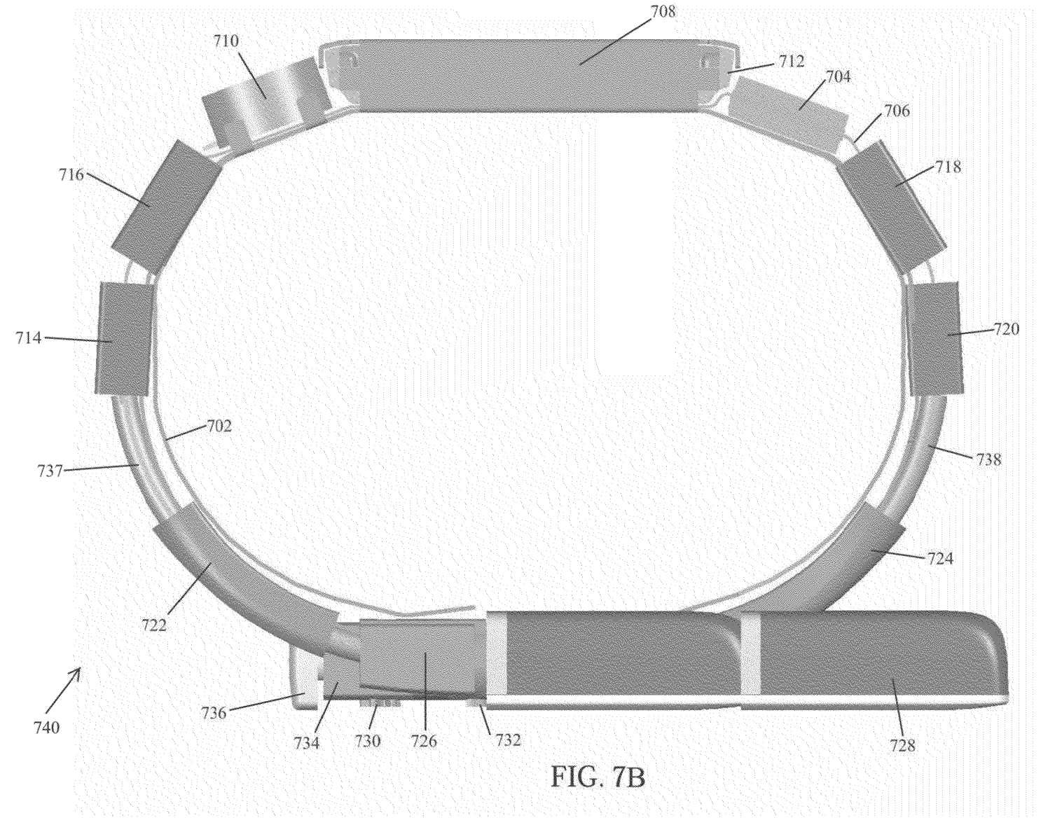

FIG. 7A illustrates a perspective view of an exemplary data-capable band configured to receive overmolding. Here, band 700 includes framework 702, covering 704, flexible circuit 706, covering 708, motor 710, coverings 714-724, plug 726, accessory 728, control housing 734, control 736, and flexible circuits 737-738. In some examples, band 700 is shown with various elements (i.e., covering 704, flexible circuit 706, covering 708, motor 710, coverings 714-724, plug 726, accessory 728, control housing 734, control 736, and flexible circuits 737-738) coupled to framework 702. Coverings 708, 714-724 and control housing 734 may be configured to protect various types of elements, which may be electrical, electronic, mechanical, structural, or of another type, without limitation. For example, covering 708 may be used to protect a battery and power management module from protective material formed around band 700 during an injection molding operation. As another example, housing 704 may be used to protect a printed circuit board assembly (“PCBA”) from similar damage. Further, control housing 734 may be used to protect various types of user interfaces (e.g., switches, buttons (e.g., control 736), lights, light-emitting diodes, or other control features and functionality) from damage. In other examples, the elements shown may be varied in quantity, type, manufacturer, specification, function, structure, or other aspects in order to provide data capture, communication, analysis, usage, and other capabilities to band 700, which may be worn by a user around a wrist, arm, leg, ankle, neck or other protrusion or aperture, without restriction. Band 700, in some examples, illustrates an initial unlayered device that may be protected using the techniques for protective overmolding as described above. Alternatively, the number, type, function, configuration, ornamental appearance, or other aspects shown may be varied without limitation.

FIG. 7B illustrates a side view of an exemplary data-capable band. Here, band 740 includes framework 702, covering 704, flexible circuit 706, covering 708, motor 710, battery 712, coverings 714-724, plug 726, accessory 728, button/switch/LED 730-732, control housing 734, control 736, and flexible circuits 737-738 and is shown as a side view of band 700. In other examples, the number, type, function, configuration, ornamental appearance, or other aspects shown may be varied without limitation.

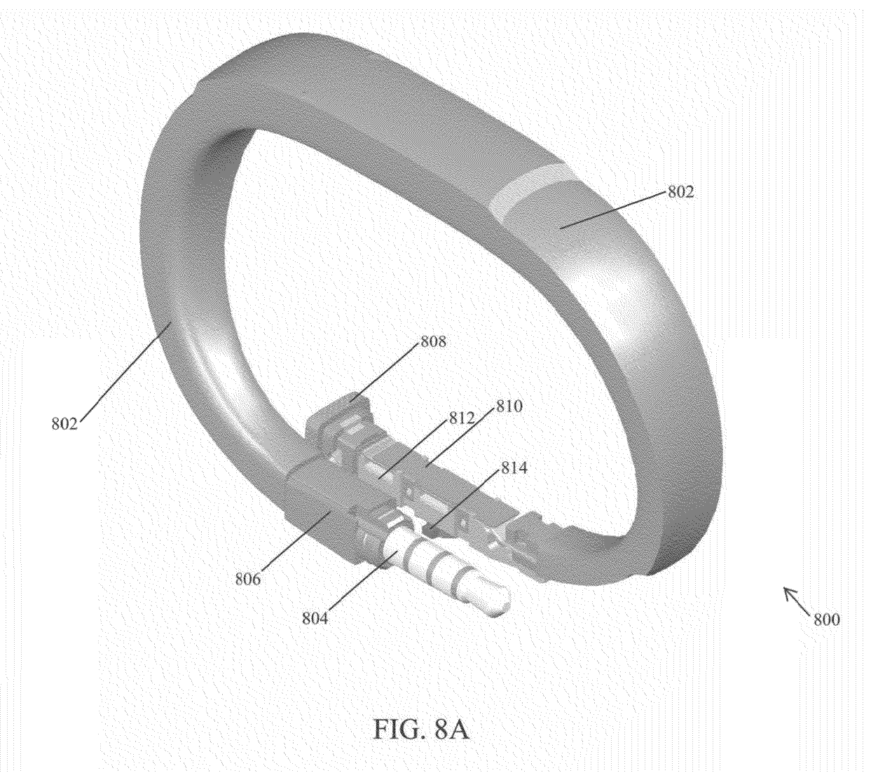

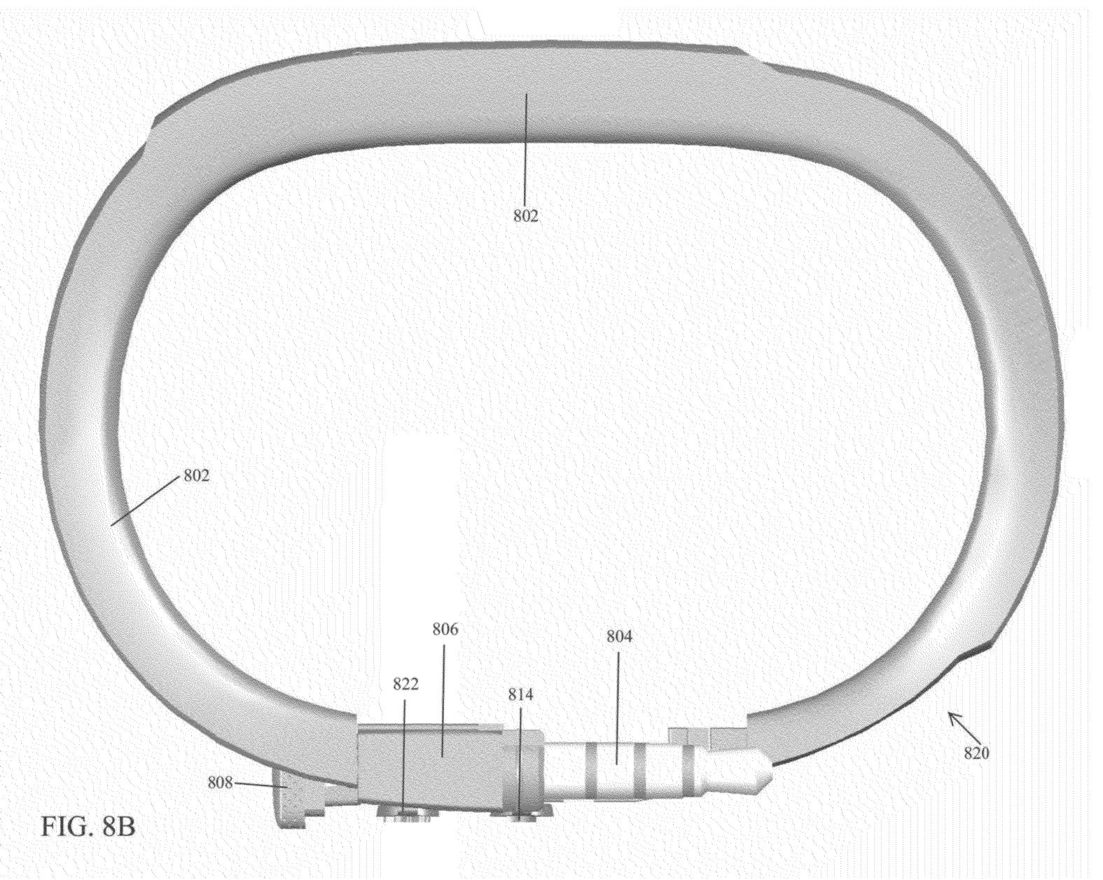

FIG. 8A illustrates a perspective of an exemplary data-capable band having a first molding. Here, an alternative band (i.e., band 800) includes molding 802, analog audio TRRS-type plug (hereafter “plug”) 804, plug housing 806, button 808, framework 810, control housing 812, and indicator light 814. In some examples, a first protective overmolding (i.e., molding 802) has been applied over band 700 (FIG. 7) and the above-described elements (e.g., covering 704, flexible circuit 706, covering 708, motor 710, coverings 714-724, plug 726, accessory 728, control housing 734, control 736, and flexible circuit 738) leaving some elements partially exposed (e.g., plug 804, plug housing 806, button 808, framework 810, control housing 812, and indicator light 814). However, internal PCBAs, flexible connectors, circuitry, and other sensitive elements have been protectively covered with a first or inner molding that can be configured to further protect band 800 from subsequent moldings formed over band 800 using the above-described techniques. In other examples, the type, configuration, location, shape, design, layout, or other aspects of band 800 may be varied and are not limited to those shown and described. For example, TRRS plug 804 may be removed if a wireless communication facility is instead attached to framework 810, thus having a transceiver, logic, and antenna instead being protected by molding 802. As another example, button 808 may be removed and replaced by another control mechanism (e.g., an accelerometer that provides motion data to a processor that, using firmware and/or an application, can identify and resolve different types of motion that band 800 is undergoing), thus enabling molding 802 to be extended more fully, if not completely, over band 800. In other examples, the number, type, function, configuration, ornamental appearance, or other aspects shown may be varied without limitation.

FIG. 8B illustrates a side view of an exemplary data-capable band. Here, band 820 includes molding 802, plug 804, plug housing 806, button 808, control housing 812, and indicator lights 814 and 822. In other examples, the number, type, function, configuration, ornamental appearance, or other aspects shown may be varied without limitation.

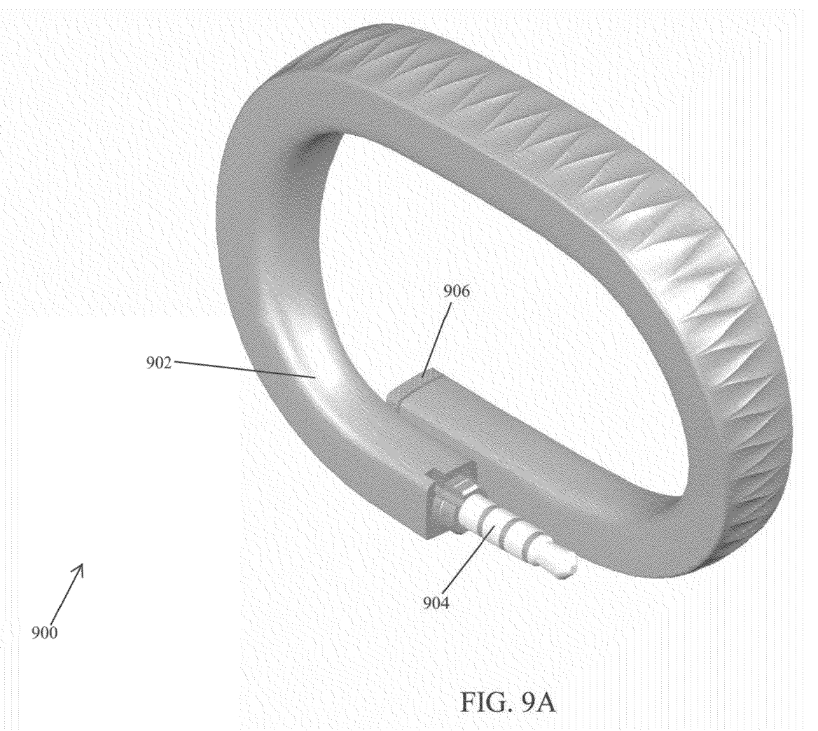

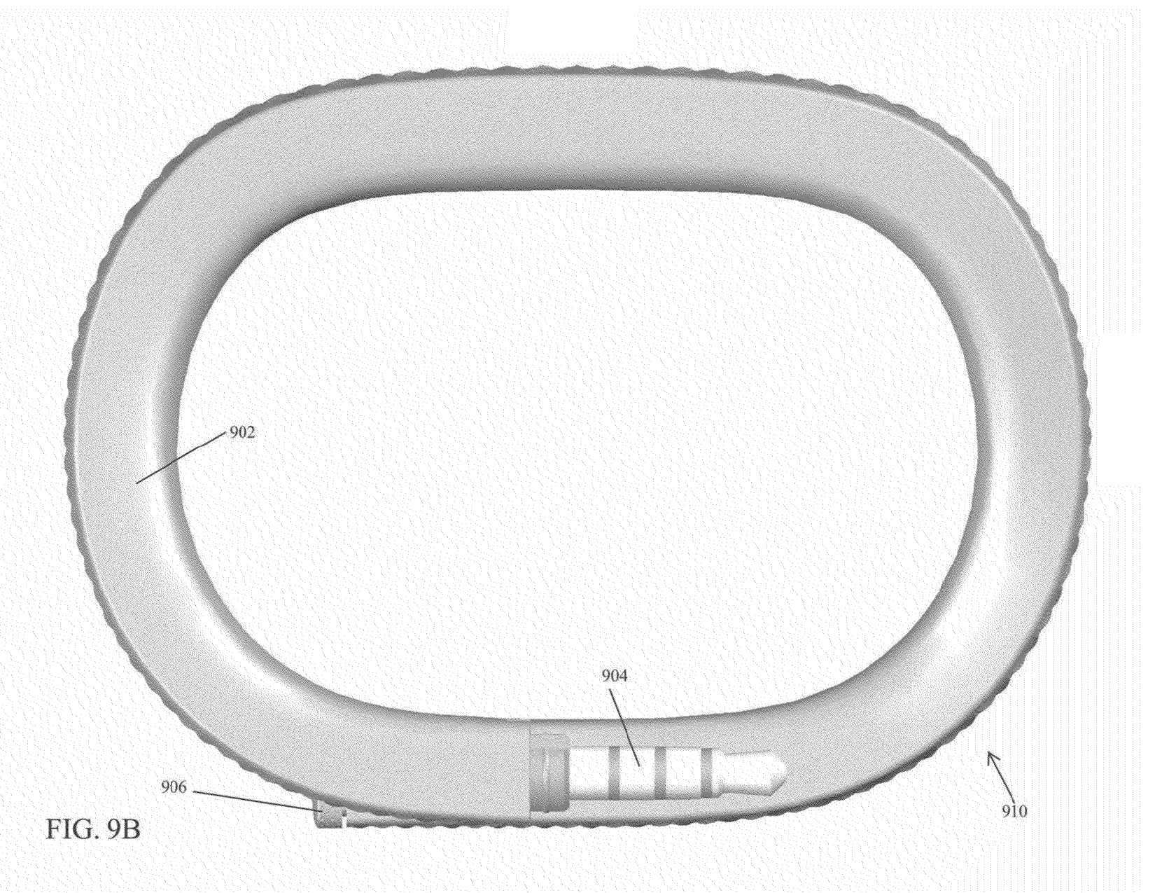

FIG. 9A illustrates a perspective view of an exemplary data-capable band having a second molding. Here, band 900 includes molding 902, plug 904, and button 906. As shown another overmolding or protective material has been formed by injection molding, for example, molding 902 over band 900. As another molding or covering layer, molding 902 may also be configured to receive surface designs, raised textures, or patterns, which may be used to add to the commercial appeal of band 900. In some examples, band 900 may be illustrative of a finished data-capable band (i.e., band 700 (FIG. 7), 800 (FIG. 8) or 900) that may be configured to provide a wide range of electrical, electronic, mechanical, structural, photonic, or other capabilities.

Here, band 900 may be configured to perform data communication with one or more other data-capable devices (e.g., other bands, computers, networked computers, clients, servers, peers, and the like) using wired or wireless features. For example, plug 900 may be used, in connection with firmware and software that allow for the transmission of audio tones to send or receive encoded data, which may be performed using a variety of encoded waveforms and protocols, without limitation. In other examples, plug 904 may be removed and instead replaced with a wireless communication facility that is protected by molding 902. If using a wireless communication facility and protocol, band 900 may communicate with other data-capable devices such as cell phones, smart phones, computers (e.g., desktop, laptop, notebook, tablet, and the like), computing networks and clouds, and other types of data-capable devices, without limitation. In still other examples, band 900 and the elements described above in connection with FIGS. 1-9, may be varied in type, configuration, function, structure, or other aspects, without limitation to any of the examples shown and described.

FIG. 9B illustrates a side view of an exemplary data-capable band. Here, band 910 includes molding 902, plug 904, and button 906. In other examples, the number, type, function, configuration, ornamental appearance, or other aspects shown may be varied without limitation.

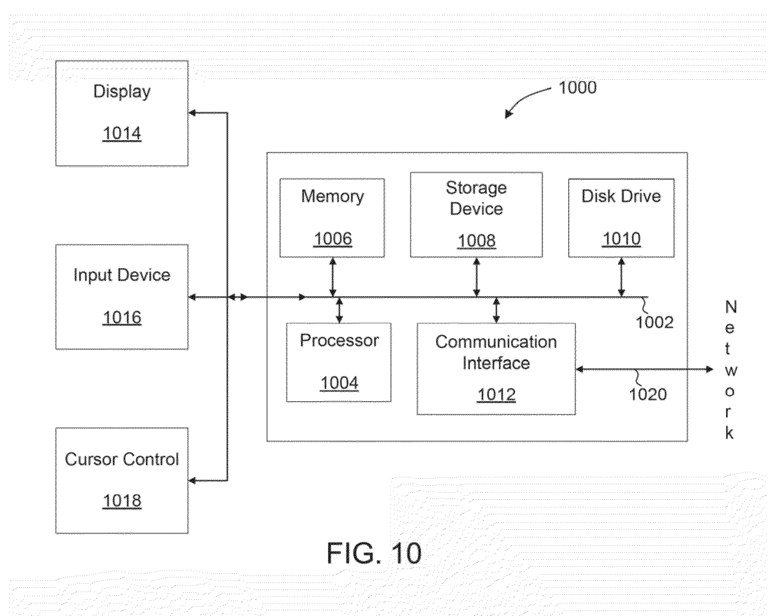

FIG. 10 illustrates an exemplary computer system suitable for use with a data-capable band. In some examples, computer system 1000 may be used to implement computer programs, applications, methods, processes, or other software to perform the above-described techniques. Computer system 1000 includes a bus 1002 or other communication mechanism for communicating information, which interconnects subsystems and devices, such as processor 1004, system memory 1006 (e.g., RAM), storage device 1008 (e.g., ROM), disk drive 1010 (e.g., magnetic or optical), communication interface 1012 (e.g., modem or Ethernet card), display 1014 (e.g., CRT or LCD), input device 1016 (e.g., keyboard), and cursor control 1018 (e.g., mouse or trackball).

According to some examples, computer system 1000 performs specific operations by processor 1004 executing one or more sequences of one or more instructions stored in system memory 1006. Such instructions may be read into system memory 1006 from another computer readable medium, such as static storage device 1008 or disk drive 1010. In some examples, hard-wired circuitry may be used in place of or in combination with software instructions for implementation.

The term “computer readable medium” refers to any tangible medium that participates in providing instructions to processor 1004 for execution. Such a medium may take many forms, including but not limited to, non-volatile media and volatile media. Non-volatile media includes, for example, optical or magnetic disks, such as disk drive 1010. Volatile media includes dynamic memory, such as system memory 1006.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1002 for transmitting a computer data signal.

In some examples, execution of the sequences of instructions may be performed by a single computer system 1000. According to some examples, two or more computer systems 1000 coupled by communication link 1020 (e.g., LAN, PSTN, or wireless network) may perform the sequence of instructions in coordination with one another. Computer system 1000 may transmit and receive messages, data, and instructions, including program, i.e., application code, through communication link 1020 and communication interface 1012. Received program code may be executed by processor 1004 as it is received, and/or stored in disk drive 1010, or other non-volatile storage for later execution.

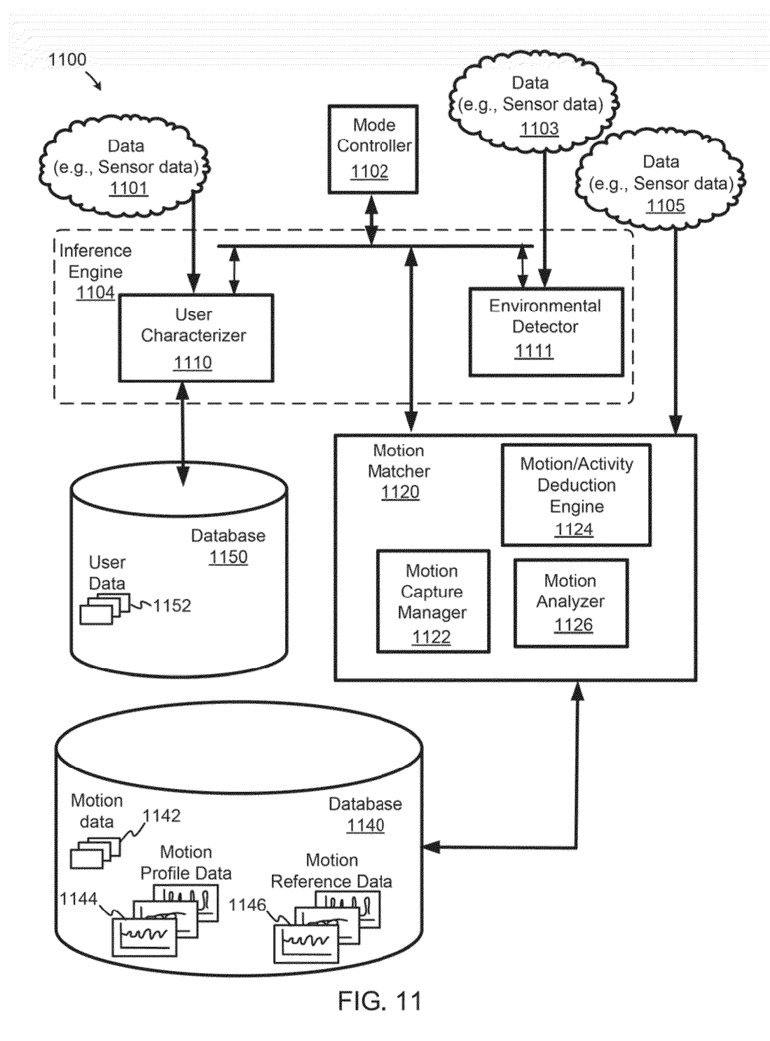

FIG. 11 depicts an exemplary inference engine of a band configured to detect an activity and/or a mode based on monitored motion. In some embodiments, inference engine 1104 of a band can be configured to detect an activity or mode, or a state of a band, as a function of at least data derived from one or more sources of data, such as any number of sensors. Examples of data obtained by the sensors include, but are not limited to, data describing motion, location, user characteristics (e.g., heart rate, body temperature, etc.), environmental characteristics (e.g., time, degree of ambient light, altitude, magnetic flux (e.g., magnetic field of the earth), or any other source of magnetic flux), GPS-generated position data, proximity to other band wearers, etc.), and data derived or sensed by any source of relevant information. Further, inference engine 1104 is configured to analyze sets of data from a variety of inputs and sources of information to identify an activity, mode and/or state of a band. In one example, a set of sensor data can include GPS-derived data, data representing magnetic flux, data representing rotation (e.g., as derived by a gyroscope), and any other data that can be relevant to inference engine 1104 in its operation. The inference engine can use positional data along with motion-related information to identify an activity or mode, among other purposes.

According to some embodiments, inference engine 1104 can be configured to analyze real-time sensor data, such as user-related data 1101 derived in real-time from sensors and/or environmental-related data 1103 derived in real-time from sensors. In particular, inference engine 1104 can compare any of the data derived in real-time (or from storage) against other types of data (regardless of whether the data is real-time or archived). The data can originate from different sensors, and can obtained in real-time or from memory as user data 1152. Therefore, inference engine 1104 can be configured to compare data (or sets of data) against each other, thereby matching sensor data, as well as other data, to determine an activity or mode.

Diagram 1100 depicts an example of an inference engine 1104 that is configured to determine an activity in which the user is engaged, as a function of motion and, in some embodiments, as a function of sensor data, such as user-related data 1101 derived from sensors and/or environmental-related data 1103 derived from sensors. Examples of activities that inference engine 1104 evaluates include sitting, sleeping, working, running, walking, playing soccer or baseball, swimming, resting, socializing, touring, visiting various locations, shopping at a store, and the like. These activities may be associated with different motions of the user, and, in particular, different motions of one or more locomotive members (e.g., motion of a user's arm or wrist) that are inherent in the different activities. For example, a user's wrist motion during running may be more “pendulum-like” in its motion pattern, whereas, the wrist motion during swimming (e.g., freestyle strokes) may be more “circular-like” in its motion pattern. Diagram 1100 also depicts a motion matcher 1120, which is configured to detect and analyze motion to determine the activity (or the most probable activity) in which the user is engaged. To further refine the determination of the activity, inference engine 1104 includes a user characterizer 1110 and an environmental detector 1111 to detect sensor data for purposes of comparing subsets of sensor data (e.g., one or more types of data) against other subsets of data. Upon determining a match between sensor data, inference engine 1104 can use the matched sensor data, as well as motion-related data, to identify a specific activity or mode. User characterizer 1110 is configured to accept user-related data 1101 from relevant sensors. Examples of user-related data 1101 include heart rate, body temperature, or any other personally-related information with which inference engine 1104 can determine, for example, whether a user is sleeping or not. Further, environmental detector 1111 is configured to accept environmental-related data 1103 from relevant sensors. Examples of environmental-related data 1103 include time, ambient temperature, degree of brightness (e.g., whether in the dark or in sunlight), location data (e.g., GPS data, or derived from wireless networks), or any other environmental-related information with which inference engine 1104 can determine whether a user is engaged in a particular activity.

A band can operate in different modes of operation. One mode of operation may be an “active mode.” Active mode can be associated with activities that involve relatively high degrees of motion at relatively high rates of change. Thus, a band enters the active mode to sufficiently capture and monitor data with such activities, with conservation of power consumption as being less critical. In this mode, a controller, such as mode controller 1102, operates at a higher sample rate to capture the motion of the band at, for example, higher rates of speed. Certain safety or health-related monitoring can be implemented in active mode, or, in response to engaging in a specific activity. For example, a controller of a band can monitor a user's heart rate against normal and abnormal heart rates to alert the user to any issues during, for example, a strenuous activity. In some embodiments, a band can be configured as set forth in FIG. 5B and user characterizer 1110 can process user-related information from sensors described in relation to FIG. 5B. Another mode of operation may be a “sleep mode.” Sleep mode can be associated with activities that involve relatively low degrees of motion at relatively low rates of change, or with particular types of motion (e.g., related to breathing, tossing and turning, snoring, and other types of motion related to sleep). Thus, a band enters the sleep mode to sufficiently capture and monitor data associated with such activities, for example, while preserving power. In some embodiments, a band can be configured as set forth in FIG. 5C and user characterizer 1110 can process user-related information from sensors described in relation to FIG. 5C. Yet another mode may be “normal mode,” in which the band operates in accordance with typical user activities, such as during work (e.g., typing, standing, sitting, carrying a light object, walking a short distance, and other activities associated with work), travel (e.g., driving, boarding a train, holding a newspaper, carrying a bag or briefcase, and other activities associated with travel), movement around the house, bathing, a daily chore (e.g., vacuuming, washing a dish, making a bed, writing a letter or e-mail, wiping a surface, and other activities associated with a daily chore), walking the dog, and other activities. A band can operate in any number of different modes, including a health monitoring mode, which can implement, for example, the features set forth in FIG. 5D, or a “social mode” of operation in which the user interacts with other users of similar bands or communication devices, and, thus, a band can implement, for example, the features set forth in FIG. 5E. Any of these modes can be entered or exited either explicitly (e.g., using motion signatures, buttons, or other forms of input, as described herein) or implicitly. In still other examples, a band may operate in different modes using different types of sensor data than those described herein.

Diagram 1100 also depicts a motion matcher 1120, which is configured to detect and analyze motion to determine the activity (or the most probable activity) in which the user is engaged. In various embodiments, motion matcher 1120 can form part of inference engine 1104 (not shown), or can have a structure and/or function separate therefrom (as shown). Regardless, the structures and/or functions of inference engine 1104, including user characterizer 1110 and environmental detector 1111, and motion matcher 1120 may cooperate to determine an activity in which the user is engaged and transmit data indicating the activity (and other related information) to a controller (e.g., a mode controller 1102) that is configured to control operation of a mode, such as an “active mode,” of the band.

Motion matcher 1120 of FIG. 11 may include a motion/activity deduction engine 1124, a motion capture manager 1122 and a motion analyzer 1126. Motion matcher 1120 can receive motion-related data 1103 from relevant sensors, including those sensors that relate to space or position and to time. Examples of such sensors include accelerometers, motion detectors, velocimeters, altimeters, barometers, or other sensors. A wide variety of sensors may be implemented to provide motion-related data 1103 to motion matcher 1120. Motion capture manager 1122 may be configured to capture portions of motion, and to aggregate those portions of motion to form an aggregated motion pattern or profile. Further, motion capture manager 1122 may be configured to store motion patterns as profiles 1144 in database 1140 for real-time or future analysis or use. As described in more detail below, these motion profiles may be used as templates for future reference, either by the user that created the profile or by other users. Motion profiles 1144 may include sets of data relating to instances of motion or aggregated portions of motion (e.g., as a function of time and space, such as expressed in X, Y, Z coordinate systems).

For example, motion capture manager 1122 may be configured to capture motion relating to the activity of walking and motion relating to running, each motion being associated with a specific profile 1144. To illustrate, consider that motion profiles 1144 of walking and running share some portions of motion in common. For example, the user's wrist motion during running and walking share a “pendulum-like” pattern over time, but differ in sampled positions of the band. During walking, the wrist and band is generally at waist-level as the user walks with arms relaxed (e.g., swinging of the arms during walking can result in a longer arc-like motion pattern over distance and time), whereas during running, a user typically raises the wrists and changes the orientation of the band (e.g., swinging of the arms during running can result in a shorter arc-like motion pattern). Motion/activity deduction engine 1124 may be configured to access profiles 1144 and deduce, for example, in real-time whether the activity is walking or running.

Motion/activity deduction engine 1124 may be configured to analyze a portion of motion and deduce the activity (e.g., as an aggregate of the portions of motion) in which the user is engaged and provide that information to the inference engine 1104, which, in turn, compares user characteristics and environmental characteristics against the deduced activity to confirm or reject the determination. For example, if motion/activity deduction engine 1124 deduces that monitored motion indicates that the user is sleeping, then the heart rate of the user, as a user characteristic, can be used to compare against thresholds in user data 1152 of database 1150 to confirm that the user's heart rate is consistent with a sleeping user. User data 1152 may also include past location data, whereby historic location data can be used to determine whether a location is frequented by a user (e.g., as a means of identifying the user). Further, inference engine 1104 may be configured to evaluate environmental characteristics, such as whether there is ambient light (e.g., darkness implies conditions for resting), the time of day (e.g., a person's sleeping times typically can be between 12 midnight and 6 am), or other related information.

In operation, motion/activity deduction engine 1124 may be configured to store motion-related data to form motion profiles 1144 in real-time (or near real-time). In some embodiments, the motion-related data can be compared against motion reference data 1146 to determine “a match” of motions. Such a match may be sufficiently similar or it may be exact, depending on the context. Motion reference data 1146, which includes reference motion profiles (i.e., motion profile templates) and patterns, may be derived by motion data captured for the user during previous activities, whereby the previous activities and motion thereof serve as a reference against which to compare. Motion reference data 1146 also may include ideal or statistically-relevant motion patterns against which motion/activity deduction engine 1124 determines a match by determining which reference profile data 1146 “best fits” the real-time motion data. As used herein, “reference motion profiles” and “motion profile templates” are used interchangeably to refer to a predetermined set of motion data. In some examples, motion/activity deduction engine 1124 can operate to determine a motion pattern, and, thus, determine an activity. Note that motion reference profile data 1146, in some embodiments, serves as a “motion fingerprint” for a user and can be unique and personal to a specific user. Therefore, motion reference profile data 1146 can be used by a controller to determine whether subsequent use of a band is by the authorized user or whether the current user's real-time motion data is a mismatch against motion reference profile data 1146. If there is mismatch, a controller can activate a security protocol responsive to the unauthorized use to preserve information or generate an alert to be communicated external to the band.

Motion analyzer 1126 may be configured to analyze motion, for example, in real-time, among other things. For example, if the user is swinging a baseball bat or golf club (e.g., when the band is located on the wrist) or the user is kicking a soccer ball (e.g., when the band is located on the ankle), motion analyzer 1126 evaluates the captured motion to detect, for example, a deceleration in motion (e.g., as a motion-centric event), which can be indicative of an impulse event, such as striking an object, like a golf ball. Motion-related characteristics, such as space and time, as well as other environment and user characteristics can be captured relating to the motion-centric event. A motion-centric event, for example, is an event that can relate to changes in position during motion, as well as changes in time or velocity. In some embodiments, inference engine 1104 stores user characteristic data and environmental data in database 1150 as user data 1152 for archival purposes, reporting purposes, or any other purpose. Similarly inference engine 1104 and/or motion matcher 1120 can store motion-related data as motion data 1142 for real-time and/or future use (e.g., as a template). According to some embodiments, stored data can be accessed by a user or any entity (e.g., a third party) to adjust the data of databases 1140 and 1150 to, for example, optimize motion profile data or sensor data to ensure more accurate results. In an example, a user may access motion profile data in database 1150. In another example, a user may adjust the functionality of inference engine 1104 to ensure more accurate or precise determinations. For example, if inference engine 1104 detects a user's walking motion as a running motion, the user may modify the behavior of the logic in the band to increase the accuracy and optimize the operation of the band. A user may make the above-described adjustments in various ways (e.g., direct programming, downloaded software modules or applications, etc.). According to other embodiments, motion profiles may be stored as templates available for access by a user or any entity (e.g., a third party) to compare and hone a user's activity motions.

FIG. 12 depicts a representative implementation of one or more bands and equivalent devices, as wearable devices, to form unique motion profiles. In diagram 1200, bands and an equivalent device are disposed on locomotive members of the user, whereby the locomotive members facilitate motion relative to and about a center point 1230 (e.g., a reference point for a position, such as a center of mass). A headset 1210 may be configured to communicate with bands 1211, 1212, 1213 and 1214 and is disposed on a body portion 1202 (e.g., the head), which is subject to motion relative to center point 1230. Bands 1211 and 1212 may be disposed on locomotive portions 1204 of the user (e.g., the arms or wrists), and bands 1213 and 1214 may be disposed on locomotive portion 1206 of the user (e.g., the legs or ankles), as shown. Also as shown, headset 1210 may be disposed at distance 1220 from center point 1230, bands 1211 and 1212 are disposed at distance 1222 from center point 1230, and bands 1213 and 1214 are disposed at distance 1224 from center point 1230. A great number of users have different values of distances 1220, 1222, and 1224. Further, different wrist-to-elbow and elbow-to-shoulder lengths for different users affect the relative motion of bands 1211 and 1212 about center point 1230, and similarly, different hip-to-knee and knee-to-ankle lengths for different users affect the relative motion of bands 1213 and 1214 about center point 1230. Moreover, a great number of users have unique gaits and styles of motion. The above-described factors, as well as other factors, may facilitate the determination of a unique motion profile for a user per activity (or in combination of a number of activities). The uniqueness of the motion patterns in which a user performs an activity enables the use of motion profile data to provide a “motion fingerprint.” A “motion fingerprint” is unique to a user and can be compared against detected motion profiles to determine, for example, whether a use of the band by a subsequent wearer is unauthorized. In some cases, unauthorized users do not typically share common motion profiles. Note that while four are shown, fewer than four can be used to establish a “motion fingerprint,” or more can be shown (e.g., a band can be disposed in a pocket or otherwise carried by the user). For example, a user can place a single bands at different portions of the body to capture motion patterns for those body parts in a serial fashion. Then, each of the motions patterns can be combined to form a “motion fingerprint.” In some cases, a single band 1211 is sufficient to establish a “motion fingerprint.” In other cases, one or more of bands 1211, 1212, 1213 and 1214 may be configured to operate with multiple users, including non-human users, such as pets or other animals.

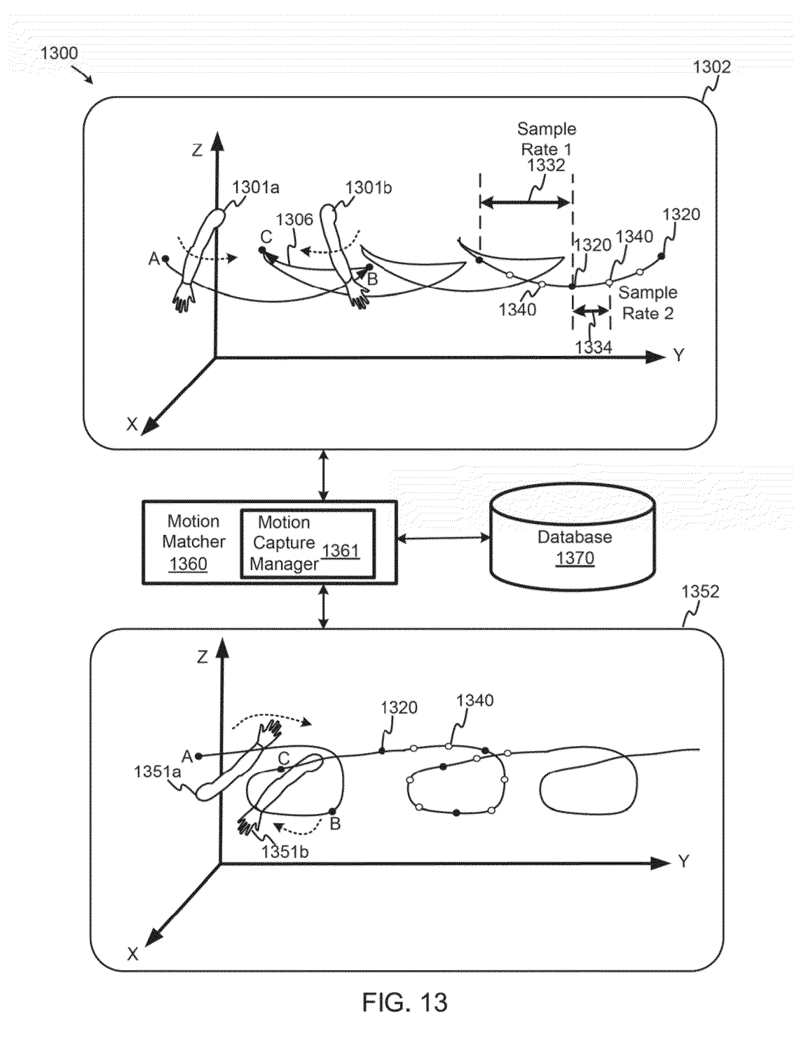

FIG. 13 depicts an example of a motion capture manager configured to capture motion and portions therefore. Diagram 1300 depicts an example of a motion matcher 1360 and/or a motion capture manager 1361, one or both of which are configured to capture motion of an activity or state of a user and generate one or more motion profiles, such as motion profile 1302 and motion profile 1352. Database 1370 is configured to store motion profiles 1302 and 1352. Note that motion profiles 1302 and 1352 are shown as graphical representation of motion data for purposes of discussion, and can be stored in any suitable data structure or arrangement. Note, too, that motion profiles 1302 and 1352 can represent real-time motion data with which a motion matcher 1360 uses to determine modes and activities.

To illustrate operation of motion capture manager 1361, consider that motion profile 1302 represents motion data captured for a running or walking activity. The data of motion profile 1302 indicates the user is traversing along the Y-axis with motions describable in X, Y, Z coordinates or any other coordinate system. The rate at which motion is captured along the Y-axis is based on the sampling rate and includes a time component. For a band disposed on a wrist of a user, motion capture manager 1361 captures portions of motion, such as repeated motion segments A-to-B and B-to-C. In particular, motion capture manager 1361 is configured to detect motion for an arm 1301a in the +Y direction from the beginning of the forward swinging arm (e.g., point A) to the end of the forward swinging arm (e.g., point B). Further, motion capture manager 1361 is configured to detect motion for arm 1301b in the −Y direction from the beginning of the backward swinging arm (e.g., point B) to the end of the backward swinging arm (e.g., point C). Note that point C is at a greater distance along the Y-axis than point A as the center point or center mass of the user has advanced in the +Y direction. Motion capture manager 1361 continues to monitor and capture motion until, for example, motion capture manager 1361 detects no significant motion (i.e., below a threshold) or an activity or mode is ended.

In some embodiments, a motion profile can be captured by motion capture manager 1361 in a “normal mode” of operation and sampled at a first sampling rate (“sample rate 1”) 1332 between samples of data 1320, which is a relatively slow sampling rate that is configured to operate with normal activities. Samples of data 1320 represent not only motion data (e.g., data regarding X, Y, and Z coordinates, time, accelerations, velocities, etc.), but can also represent or link to user related information captured at those sample times. According to some embodiments, motion matcher 1360 analyzes the motion, and, if the motion relates to an activity associated with an “active mode,” motion matcher 1360 signals to a controller, such as a mode controller, to change modes (e.g., from normal to active mode). During active mode, the sampling rate increases to a second sampling rate (“sample rate 2”) 1334 between samples of data 1320 (e.g., as well as between a sample of data 1320 and a sample of data 1340). An increased sampling rate can facilitate, for example, a more accurate set of captured motion data. To illustrate the above, consider that a user is sitting or stretching prior to a work out. The user's activities likely are occurring in a normal mode of operation. But once motion data of profile 1302 is detected, a motion/activity deduction engine can deduce the activity of running, and then can infer the mode ought to be the active mode. The logic of the band then can place the band into the active mode. Therefore, the band can change modes of operation implicitly (i.e., explicit actions to change modes need not be necessary). In some cases, a mode controller can identify an activity as a “running” activity, and then invoke activity-specific functions, such as an indication (e.g., a vibratory indication) to the user every one-quarter mile or 15 minute duration during the activity.

FIG. 13 also depicts another motion profile 1352. Consider that motion profile 1352 represents motion data captured for swimming activity (e.g., using a freestyle stroke). Similar to profile 1302, the motion pattern data of motion profile 1352 indicates the user is traversing along the Y-axis. The rate at which motion is captured along the Y-axis is based on the sampling rate of samples 1320 and 1340, for example. For a band disposed on a wrist of a user, motion capture manager 1361 captures the portions of motion, such as motion segments A-to-B and B-to-C. In particular, motion capture manager 1361 is configured to detect motion for an arm 1351a in the +Y direction from the beginning of a forward arc (e.g., point A) to the end of the forward arc (e.g., point B). Further, motion capture manager 1361 is configured to detect motion for arm 1351b in the −Y direction from the beginning of reverse arc (e.g., point B) to the end of the reverse arc (e.g., point C). Motion capture manager 1361 continues to monitor and capture motion until, for example, motion capture manager 1361 detects no significant motion (i.e., below a threshold) or an activity or mode is ended.

In operation, a mode controller can determine that the motion data of profile 1352 is associated with an active mode, similar with the above-described running activity, and can place the band into the active mode, if it is not already in that mode. Further, motion matcher 1360 can analyze the motion pattern data of profile 1352 against, for example, the motion data of profile 1302 and conclude that the activity associated with the data being captured for profile 1352 does not relate to a running activity. Motion matcher 1360 then can analyze profile 1352 of the real-time generated motion data, and, if it determines a match with reference motion data for the activity of swimming, motion matcher 1360 can generate an indication that the user is performing “swimming” as an activity. Thus, the band and its logic can implicitly determine an activity that a user is performing (i.e., explicit actions to specify an activity need not be necessary). Therefore, a mode controller then can invoke swimming-specific functions, such as an application to generate an indication (e.g., a vibratory indication) to the user at completion of every lap, or can count a number of strokes. In some embodiments, motion matcher 1360 and/or a motion capture manager 1361 can be configured to implicitly determine modes of operation, such as a sleeping mode of operation (e.g., the mode controller, in part, can analyze motion patterns against a motion profile that includes sleep-related motion data) (not shown). Motion matcher 1360 and/or a motion capture manager 1361 also may be configured to determine an activity out of a number of possible activities.

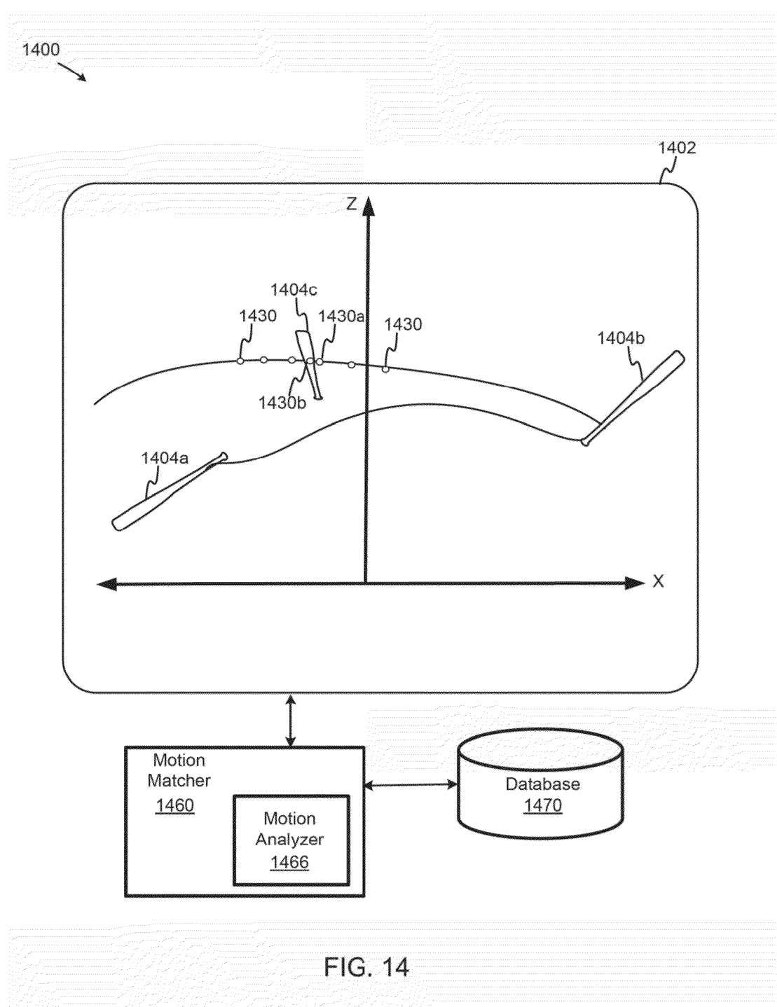

FIG. 14 depicts an example of a motion analyzer configured to evaluate motion-centric events. Diagram 1400 depicts an example of a motion matcher 1460 and/or a motion analyzer 1466 for capturing motion of an activity or state of a user and generating one or more motion profiles, such as a motion profile 1402. To illustrate, consider that motion profile 1402 represents motion data captured for an activity of swinging a baseball bat 1404. The motion pattern data of motion profile 1402 indicates the user begins the swing at position 1404a in the −Y direction. The user moves the band and the bat to position 1404b, and then swings the bat toward the −Y direction when contact is made with the baseball at position 1404c. Note that the set of data samples 1430 includes data samples 1430a and 1430b at relatively close proximity to each other in profile 1402. This indicates a deceleration (e.g., a slight, but detectable deceleration) in the bat when it hits the baseball. Thus, motion analyzer 1466 can analyze motion to determine motion-centric events, such as striking a baseball, striking a golf ball, or kicking a soccer ball. Data regarding the motion-centric events can be stored in database 1470 for additional analysis or archiving purposes, for example.

In some examples, multiple motion profiles (e.g., motion profiles 1302, 1352 and 1402) may be created for an activity type. For example, different motion profiles may be created for various types of running (e.g., a light jog, a sprint, short distance, long distance, competitive running, leisurely running, etc.). In other examples, different motion profiles may be created for different swim strokes, riding different types of bicycles (e.g., mountain vs. road), different swings of a bat, swings of different golf clubs, etc. Motion reference data 1146 may include reference motion profiles or patterns for all of these variances for each activity type.

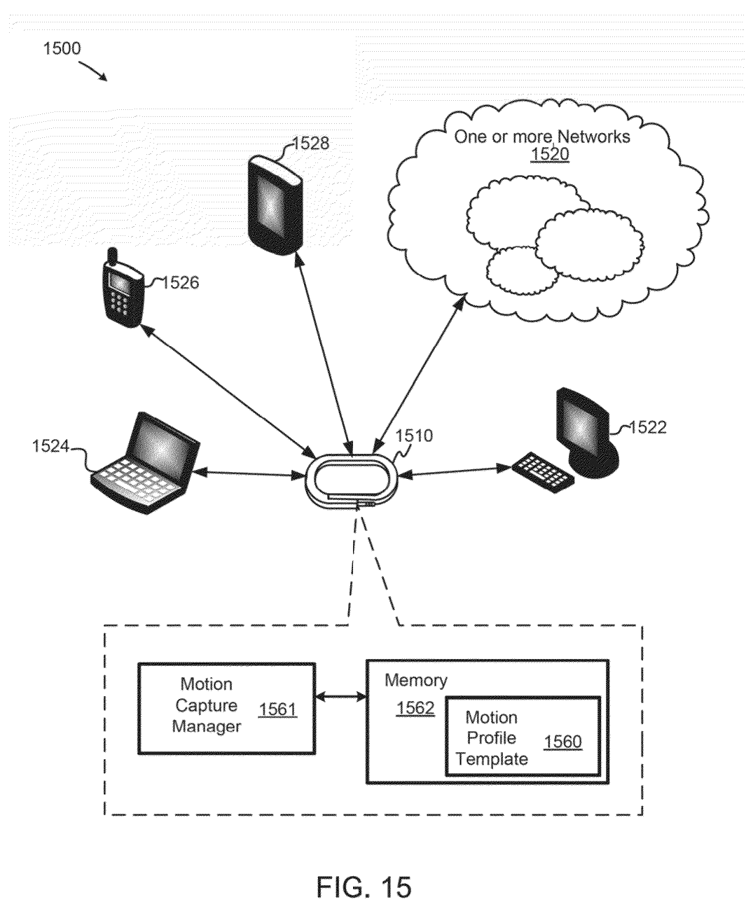

FIG. 15 illustrates an exemplary data-capable band system configured to create and share motion profile templates. System 1500 includes band 1510, one or more networks 1520, computer 1522, laptop 1524, mobile communications device 1526, and mobile computing device 1528. The elements in system 1500 may be implemented as described above with respect to corresponding elements in FIG. 1. Band 1510 is depicted as including motion capture manager 1561 and memory 1562, which includes motion profile template 1560. In some examples, memory 1562 may be implemented to store multiple motion profile templates. The elements in band 1510 also may be implemented as described above with respect to corresponding elements in FIGS. 11, 13 and 14. In some examples, a user may choose to store a motion profile as motion profile template 1560. For example, a user wearing band 1510 may have a particularly successful golf swing for a particular hole at a particular golf course. Motion capture manager 1561 may capture that golf swing as motion profile template 1560 and store it in memory 1562 for future reference. The user may measure and compare future golf swings against motion profile template 1560. In some examples, band 1510 may share that stored motion profile template with any of the other devices or networks in system 1500, or with another band (not shown). Band 1510 may do so using any wired or wireless communication options, as described in more detail above. In some examples, band 1510 may share this information with other users through applications implemented on any of the data and communications capable devices depicted in system 1500 (e.g., networks 1520, computer 1522, laptop 1524, mobile communications device 1526, and mobile computing device 1528). The other users may then download motion profile template 1560 onto their bands (not shown), and use motion profile template 1560 as a reference for their golf swings.

Likewise, a user wearing band 1510 may obtain (e.g., download) motion profile templates created by other users onto band 1510 to use as a reference for their own activities. For example, a user may obtain a motion profile template created by an instructor of, expert in, or professional of, an activity (e.g., a tennis instructor or professional athlete). In another example, friends or colleagues may share motion profile templates for competitions associated with any sport or activity (e.g., golfing, running, swimming, cycling, driving, walking, climbing, typing, sleeping). In yet other examples, users may share motion profile templates for instructional or recreational uses.

In other embodiments, expert, ideal or instructional motion profile templates may be provided through an application (e.g., software application, online store or marketplace, etc.) (not shown). In some examples, expert, ideal or instructional motion profile templates may be implemented with a feedback and/or reward system, which may offer a user incentives (e.g., points, real or virtual coins, gifts, etc.), encouragement, or offers (e.g., discounts on products or services related to the activity, access to exclusive events, etc.) associated with a user's improvement in reference to a motion profile template. The application may be implemented on any of the data and communications capable devices depicted in system 1500 (e.g., networks 1520, computer 1522, laptop 1524, mobile communications device 1526, and mobile computing device 1528). In some examples, the application may enable the upload of motion profile templates for sharing. In other examples, an application may enable the creation of motion profile templates using textual or other human-readable input. As used herein, “human-readable” refers to any text, graphic, noise, texture, or other format that may be sensed (e.g., read, seen, felt, heard, or otherwise sensed) by a human.

In other embodiments, motion profile templates may be used to monitor and/or correct behaviors. For example, motion profile template 1560 may be implemented with other modules, programs or applications (not shown) to detect an alcoholic's drinking habit or a smoker's smoking habit. In some examples, band 1510 may be configured to provide negative feedback when it determines that a user is drinking alcohol or smoking. Band 1510 also may be configured to provide positive feedback when a user goes for certain periods of time without drinking alcohol or smoking. In still other embodiments, band 1510 may be used with the exemplary identification and security systems described below to control a variety of devices personal to the user of band 1510.

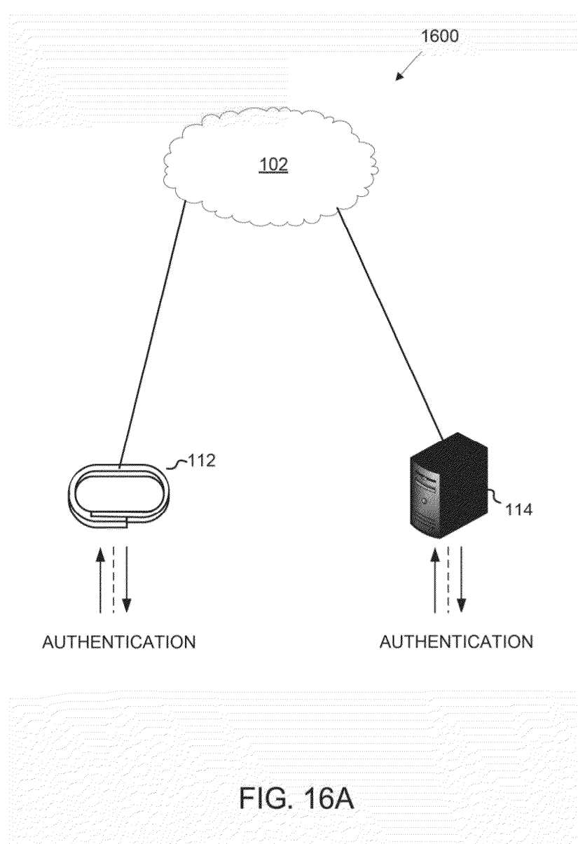

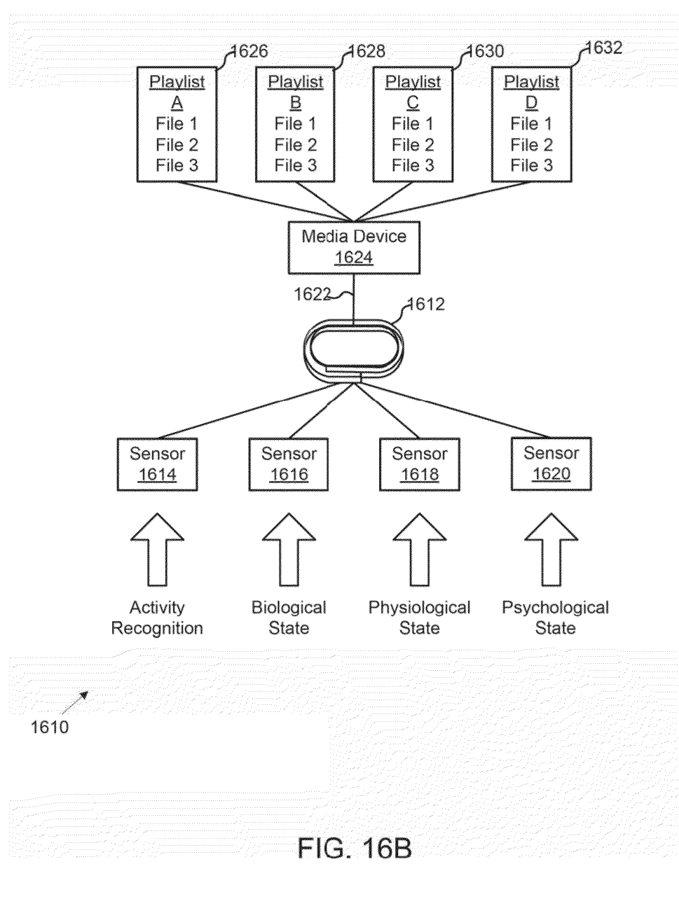

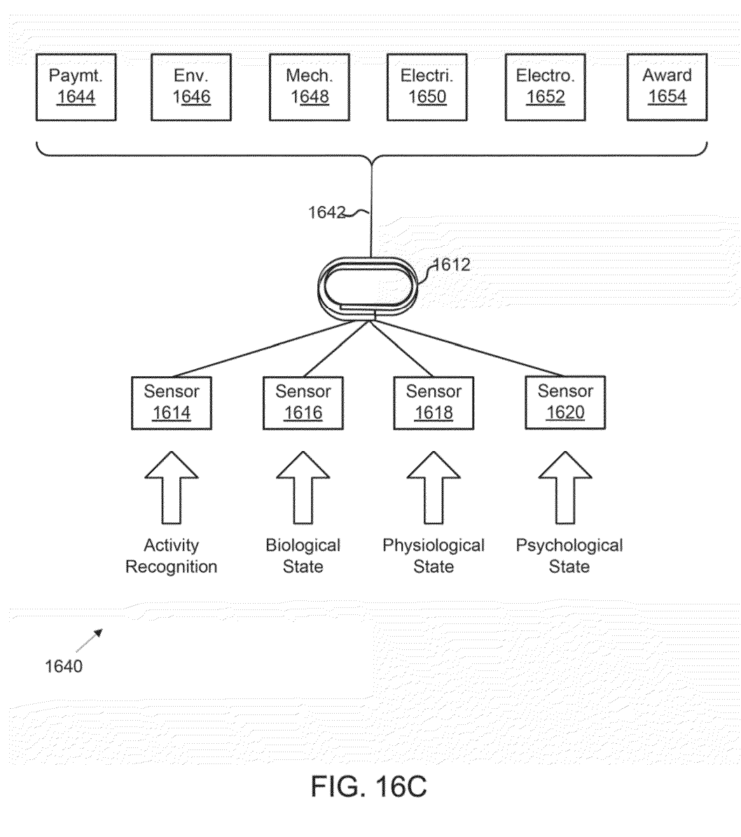

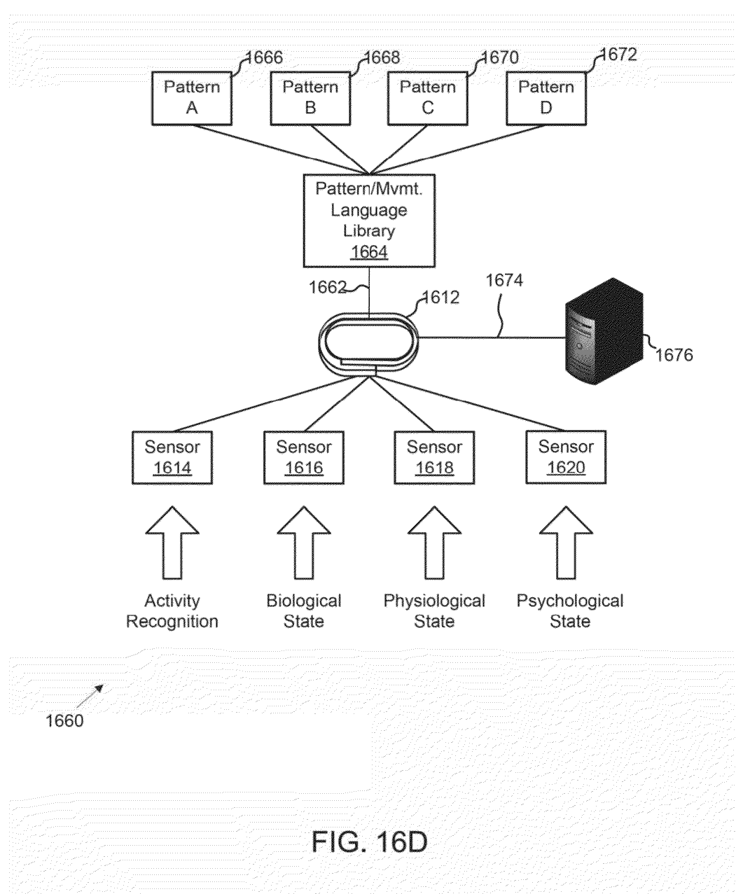

FIG. 16A illustrates an exemplary system for wearable device data security. Exemplary system 1600 comprises network 102, band 112, and server 114. In some example, band 112 may capture data that is personal, sensitive, or confidential, as described herein. In some examples, security protocols and algorithms, as described herein, may be implemented on band 112 to authenticate a user's identity and authorize access to band 112. As used herein, “authenticate” or “authentication” refers to confirming, or the confirmation of, a user's identity. Examples of security protocols and algorithms include authentication, encryption, encoding, private and public key infrastructure, passwords, checksums, hash codes and hash functions (e.g., SHA, SHA-1, MD-5, and the like), or others may be used to prevent undesired access to data captured by band 112. In other examples, authentication of a user's identity for band 112 may be implemented differently. This authentication may be implemented to prevent unwanted use or access by others. In other examples, the security protocols and algorithms may be performed by server 114, in which case band 112 may communicate with server 114 via network 102 to authenticate a user's identity. Use of the band to capture, evaluate or access a user's data, as described herein, may be predicated on authentication of the user's identity.

In some examples, band 112 may identify of a user by the user's unique pattern of behavior or motion. Band 112 may capture and evaluate data from a user to create a unique key personal to the user (e.g., based upon a user's characteristic motion). In some examples, the key may be associated with an individual user's physical attributes, including gait, biometric or physiological signatures (e.g., resting heart rate, skin temperature, salinity of emitted moisture, etc.), or any other sets of data that may be captured by band 112, as described in more detail above. In some examples, the key may be based upon a set of physical attributes that are known in combination to be unique to a user. Once the key is created based upon the predetermined, or pre-programmed, set of physical attributes, it may be used in an authentication process to authenticate a user's identity, and to prevent access to, or capture and evaluation of, data by an unauthorized user. For example, if an unauthorized user puts on band 112 and starts performing an activity, band 112 may be unable to authenticate use by this unauthorized user, and may shut off, or otherwise enter a locked mode in which band 112 does not collect data, and data stored in band 112 may not be accessed (e.g., downloaded, viewed, or otherwise accessed).

In some examples, authentication using the key may be carried out directly by band 112. In other examples, band 112 may be used with other bands (not shown) that may be owned by the same individual (i.e., user) to authenticate a user's identity. For example, multiple bands that are owned by the same individual may be configured for different sensors or types of activities, but may also be configured to share data with each other, or otherwise work together, to carry out an authentication of a user's identity. In order to prevent unauthenticated or unauthorized individuals from accessing a given user's data, band 112 may be configured using various types of authentication, identification, or other security techniques among one or more bands, including for example band 112. As an example, band 112 may be in direct data communication with other bands (not shown) or indirectly through an authentication system or service, for example implemented using server 114. In still other examples, band 112 may send data to server 114, which in turn carries out an authentication and returns a prompt, or other notification, to band 112 to unlock, or otherwise provide access to, band 112 for use. In other examples, data security and identity authentication for band 112 may be implemented differently.

FIG. 16B illustrates an exemplary system for media device content management using sensory input. Here, system 1660 includes band 1612, sensors 1614-1620, data connection 1622, media device 1624, and playlists 1626-1632. As used throughout this description, band 1612 may also be referred to interchangeably as a “wearable device.” Sensors 1614-1620 may be implemented using any type of sensor such as a 2 or 3-axis accelerometer, temperature, humidity, barometric pressure, skin resistivity (i.e., galvanic skin response (GSR)), pedometer, or any other type of sensor, without limitation. Data connection 1622 may be implemented as any type of wired or wireless connection using any type of data communication protocol (e.g., Bluetooth, wireless fidelity (i.e., WiFi), LAN, WAN, MAN, near field communication (NFC), or others, without limitation) between band 1612 and media device 1624. Data connection 1622 may be configured to transfer data bi-directionally or in a single direction between media device 1624 and band 1612. In some examples, data connection 1622 may be implemented by using a 3.5 mm audio jack (e.g., TRRS-type, TRS-type, or other type of connector) that connects to an appropriate plug (i.e., outlet) and transmits electrical signals that may be interpreted for transferring data. Alternatively, a wireless radio, transmitter, transceiver, or the like may be implemented with band 1612. In some examples, when a motion is detected via an installed accelerometer on the band 1612, a transmission of a control signal to media device 1624 may be initiated to, for example, begin playing playlist 1630, change from playlist 1630 to another playlist (e.g., playlists 1626-1628 or 1632), forward to another song on playlist 1630, and the like.

As shown, media device 1624 may be any type of device that is configured to display, play, interact, show, or otherwise present various types of media, including audio, visual, graphical, images, photographical, video, rich media, multimedia, or a combination thereof, without limitation. Examples of media device 1624 may include audio playback devices (e.g., players configured to play various formats of audio and video files including .mp3, .wav, and others, without limitation), connected or wireless (e.g., Bluetooth, WiFi, WLAN, and others, as described herein) speakers, radios, audio devices installed on portable, desktop, or mobile computing devices, and other devices. In some examples, playlists 1626-1632 may be configured to play various types of files of various formats, as representatively illustrated by “File 1, File 2, File 3” in association with each playlist. Each file on a given playlist may be any type of media and played using various types of formats or applications implemented on media device 1624.

As an example, sensors 1614-1620 may detect various types of inputs locally (i.e., on band 1612) or remotely (i.e., on another device that is in data communication with band 1612) such as an activity or motion (e.g., running, walking, swimming, jogging, jumping, shaking, turning, cycling, or others), a biological state (e.g., healthy, ill, diabetic, awake, asleep, or others), a physiological state (e.g., normal gait, limping, injured, sweating, high heart rate, high blood pressure, or others), or a psychological state (e.g., happy, depressed, angry, and the like). Other types of inputs may be sensed by sensors 1614-1620, which may be configured to gather data and transmit that information to an application that uses the data to infer various conclusions related to the above-described states or activities, among others. In some examples, each of sensors 1614-1620 may comprise a plurality, or a set, of individual sensors, each configured to capture data associated with a particular parameter associated with an activity, a biological state, a physiological state, or a psychological state. Based on the data gathered by sensors 1614-1620 and, in some examples, user or system-specified parameters, band 1612 may be configured to generate control signals (e.g., electrical or electronic signals that are generated at various types or amounts of voltage in order to produce, initiate, trigger, or otherwise cause certain actions or functions to occur). For example, data may be transferred from sensors 1614-1620 to band 1612 indicating that a user has started running. Band 1612 may be configured to generate a control signal to media device 1624 over data connection 1622 to initiate playing files in a given playlist in order. A shake of a user's wrist, for example, in a given direction or axis may cause band 1612 to generate a different control signal that causes media device 1624 to change the play order, to change files, to forward to another file, or to initiate some other action. In some examples, a given movement (e.g., a user shakes her wrist on which band 1612 is worn) may be resolved into data associated with motion occurring along each of 3-different axes. Band 1612 may be configured to detect motion using an accelerometer (not shown), which then resolves the detected motion into data associated with three separate axes of movement, translated into data or electrical control signals that may be stored in a memory that is local and/or remote to band 1612. Further, the stored data of a given motion may be associated with a specific action such that, when detected, control signals may be generated by band 1612 and sent over data connection 1622 to media device 1624 or other types of devices, without limitation.

As another example, if sensor 1616 detects that a user is lying prone and her heart rate is slowing (e.g., decelerating towards a previously-recorded resting heart rate), a control signal may be generated by band 1612 to begin playback of a song appropriate for bedtime (e.g., Brahms' Lullaby, another lullaby, or other desired bedtime song) using, for example, a Bluetooth-connected headset speaker (i.e., media device 1624). In yet another example, if sensor 1618 detects a physiological state change (e.g., a user is walking with a gait or limp as opposed to normally observed physiological behavior), media device 1624 may be controlled by band 1612 to initiate playback of a file on a graphical user interface of a connected device (e.g., a mobile computing or communications device) that provides a tutorial on running or walking injury treatment, recovery and/or prevention. As yet another example, if sensor 1620 detects one or more parameters that a user is happy (e.g., sensor 1620 detects an accelerated, but regular heart rate, rapid or erratic movements, increased body temperature, increased speech levels, and the like), band 1612 may send a control signal to media device 1624 to display an inquiry as to whether the user wishes to hear songs played from her “happy playlist” (not shown). The above-described examples are provided for purposes of illustrating the use of managing various types of media and media content using band 1612, but many others may be implemented without restriction to those provided.

FIG. 16C illustrates an exemplary system for device control using sensory input. Here, system 1640 includes band 1612, sensors 1614-620, data connection 1642, and device types 1644-1654. Those elements shown that are like-named and numbered may be designed, implemented, or configured as described above or differently. As shown, the detection by band 1612 of a given activity, biological state, physiological state, or psychological state may be gathered as data from sensors 1614-1620 and used to generate various types of control signals. Control signals, in some examples, may be transmitted via a wired or wireless data connection (e.g., data connection 1642) to one or multiple device types 1644-1654 that are in data communication with band 1612. Device types 1644-1654 may be any type of device, apparatus, application, or other mechanism that may be in data connection with, coupled to (indirectly or directly), paired (e.g., via Bluetooth or another data communication protocol), or otherwise configured to receive control signals from band 1612.

As shown, band 1612 may send control signals to various types of devices (e.g., device types 1644-1654), including payment systems (1644), environmental (1646), mechanical (1648), electrical (1650), electronic (1652), award (1654), and others, without limitation. In some examples, band 1612 may be associated with an account to which a user may link a credit card, debit card, or other type of payment account that, when properly authenticated, allows for the transmission of data and control signals (not shown) over data connection 1642 to payment system (i.e., device) 1644. In other examples, band 1612 may be used to send data that can be translated or interpreted as control signals or voltages in order to manage environmental control systems (e.g., heating, ventilation, air conditioning (HVAC), temperature, air filter (e.g., hepa, pollen, allergen), humidify, and others, without limitation). Input detected from one or more of sensors 1614-1620 may be transformed into data received by band 1612. Using firmware, application software, or other user or system-specified parameters, when data associated with input from sensors 1614-1620 are received, control signals may be generated and sent by band 1612 over data connection 1642 to environmental control system 1646, which may be configured to implement a change to one or more environmental conditions within, for example, a residential, office, commercial, building, structural, or other type of environment. As an example, if sensor 1612 detects that a user wearing band 1612 has begun running and sensor 1618 detects a rise in one or more physiological conditions, band 1612 may generate control signals and send these over data connection 1642 to environmental control system 1646 to lower the ambient air temperature to a specified threshold (as input by a user into an account storing a profile associated with environmental conditions he prefers for running (or another type of activity)) and decreasing humidity to account for increased carbon dioxide emissions due to labored breathing. As another example, sensor 1616 may detect that a given user is pregnant due to the detection of an increase in various types of hormonal levels, body temperature, and other biochemical conditions. Using this input against comparing the user's past preferred ambient temperature ranges, band 1612 may be configured to generate, without user input, one or more control signals that may be sent to operate electrical motors that are used to open or close window shades and mechanical systems that are used to open or close windows in order to adjust the ambient temperature inside her home before arriving from work. As a further example, sensor 1618 may detect that a user has been physiologically confined to a sitting position for 4 hours and sensor 1620 has received input indicating that the user is in an irritated psychological state due to an audio sensor (not shown, but implementable as sensor 1620) detecting increased noise levels (possibly, due to shouting or elevating voice levels), a temperature sensor (not shown) detecting an increase in body temperature, and a galvanic skin response sensor (not shown) detecting changes in skin resistivity (i.e., a measure of electrical conductivity of skin). Subsequently, band 1612, upon receiving this input, may compare this data against a database (either in firmware or remote over data connection 1642) and, based upon this comparison, send a control signal to an electrical system to lower internal lighting and another control signal to an electronic audio system to play calming music from memory, compact disc, or the like.

As another example, a user may have an account associated with band 1612 and enrolls in a participatory fitness program that, upon achieving certain milestones, results in the receipt of an award or promotion. For example, sensor 1614 may detect that a user has associated his account with a program to receive a promotional discount towards the purchase of a portable Bluetooth communications headset. However, the promotion may be earned once the user has completed, using band 1612, a 10 kilometer run at an 8-minute and 30-second per mile pace. Upon first detecting the completion of this event using input from, for example, a GPS sensor (not shown, but implementable as sensor 1614), a pedometer, a clock, and an accelerometer, band 1612 may be configured to send a signal or data via a wireless connection (i.e., data connection 1642) to award system 1654, which may be configured to retrieve the desired promotion from another database (e.g., a promotions database, an advertisement server, an advertisement network, or others) and then send the promotion electronically back to band 1612 for further display or use (e.g., redemption) on a device in data connection with band 1612 (not shown). Other examples of the above-described device types and other device types not shown or described may be implemented and are not limited to those provided.

FIG. 16D illustrates an exemplary system for movement languages in wearable devices. Here, system 1660 includes band 1612, sensors 1614-1620, data connection 1622, pattern/movement language library (i.e., pattern library) 1664, movement patterns (i.e., patterns) 1666-1672, data connection 1674, and server 1676. In some examples, band 1612 may be configured to compile a “movement language” that may be stored in pattern library 1664, which can be either local (i.e., in memory on band 1612) or remote (i.e., in a database or other data storage facility that is in data connection with band 1612, either via wired or wireless data connections). As used herein, a “movement language” may refer to the description of a given movement as one or more inputs (e.g., sensory, manual, or other inputs) that may be transformed into a discrete set of data that, when observed again, can be identified as correlating to a given movement. In some examples, a movement may be described as a collection of one or more motions. In other examples, biological, psychological, and physiological states or events may also be recorded in pattern library 1664. These various collections of data may be stored in pattern library 1664 as patterns 1666-1672. In some examples, a movement or pattern (e.g., patterns 1666-1672) may be unique to a user. In other examples, a movement or pattern (e.g., patterns 1666-1672) may be common or characteristic to a group of users (e.g., male, female, tall, short, old, young, athletic, obese, paraplegic, runner, swimmer, cyclist, and other groups).

A movement, when detected by an accelerometer (not shown) on band 1612, may be associated with a given data set and used, for example, to perform one or more functions when detected again. Parameters may be specified (i.e., by either a user or system (i.e., automatically or semi-automatically generated)) that also allow for tolerances to determine whether a given movement falls within a given category (e.g., jumping may be identified as a set of data that has a tolerance of +/−0.5 meters for the given individual along a z-axis as input from a 3-axes accelerometer).

Using the various types of sensors (e.g., sensors 1614-1620), different movements, motions, moods, emotions, physiological, psychological, or biological events can be monitored, recorded, stored, compared, and used for other functions by band 1612. Further, movements may also be downloaded from a remote location (e.g., server 1676) to band 1612. Input provided by sensors 1614-1620 and resolved into one or more of patterns 1666-1672 and used to initiate or perform one or more functions, such as authentication (FIG. 16A), playlist management (FIG. 16B), device control (FIG. 16C), among others. In other examples, systems 1610, 1640, 1660 and the respective above-described elements may be varied in design, implementation, configuration, function, structure, or other aspects and are not limited to those provided.

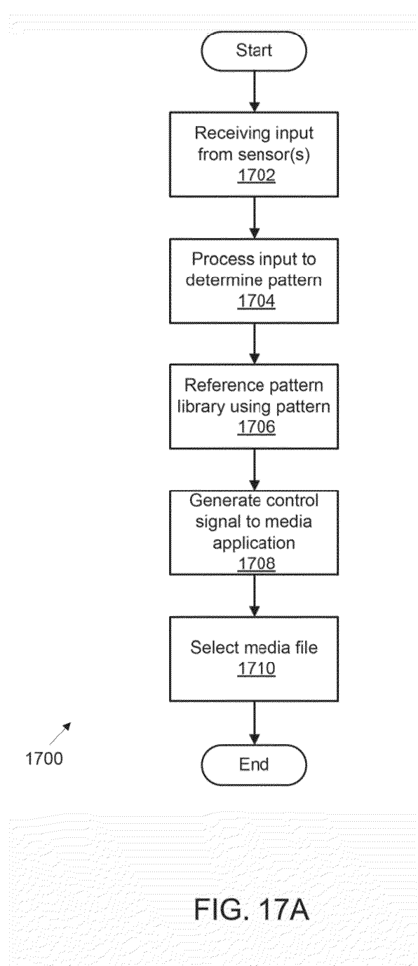

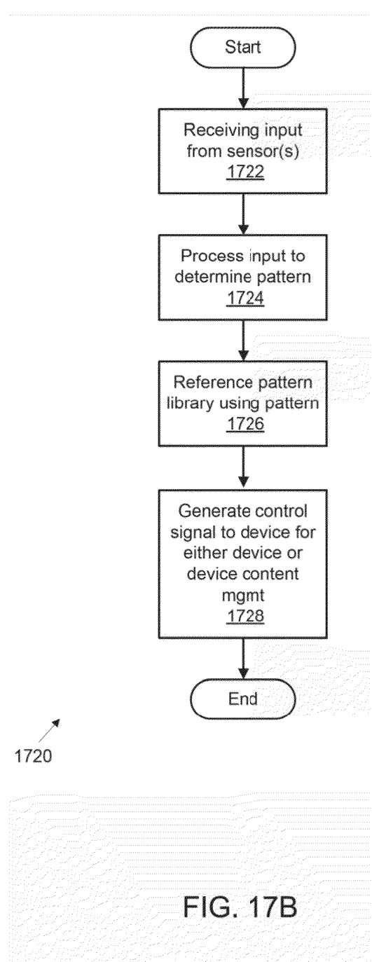

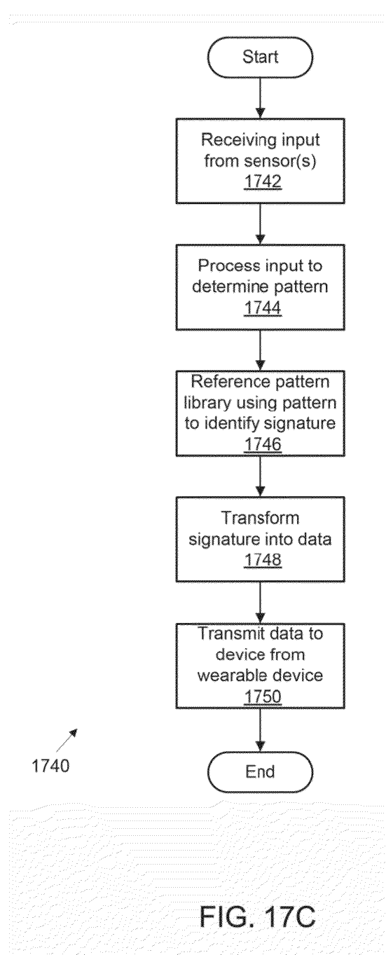

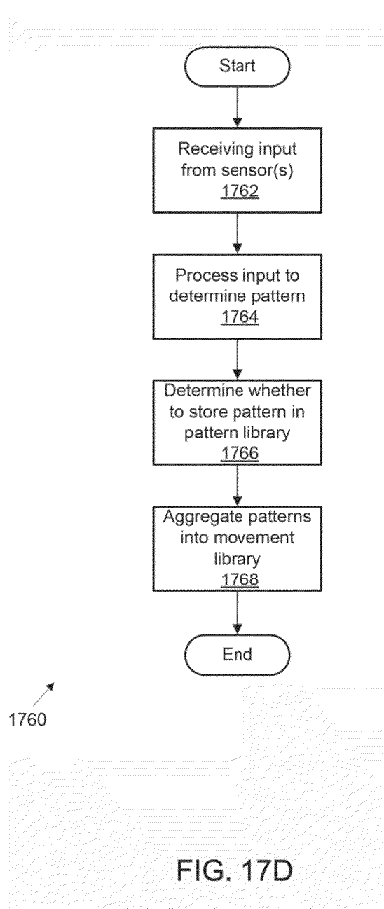

FIG. 17A illustrates an exemplary process for media device content management using sensory input. Here, process 1700 begins by receiving an input from one or more sensors that may be coupled to, integrated with, or are remote from (i.e., distributed on other devices that are in data communication with) a wearable device (1702). The received input is processed to determine a pattern (1704). In some examples, processing received sensory input may include aggregating the input into a set of inputs, categorizing the input into various categories of data, parsing the input, running an algorithm on the input, copying the input, tagging the input, or otherwise processing the input, without limitation. Once a pattern has been determined, then a compare, lookup, or other reference operation may be performed against a pattern library (i.e., a database or other storage facility configured to store data associated with one or more patterns) (1706). As used herein, “pattern library” may be used to store patterns associated with movements, motion, moods, states, activities, events, or any other grouping of data associated with a pattern as determined by evaluating input from one or more sensors coupled to a wearable device (e.g., band 104 (FIG. 1), and others). For example, a pattern associated with walking may comprise a set, or grouping, of sensory data corresponding to a movement or other parameter (e.g., physiological, biological, environmental, contextual) associated with walking (e.g., an arm movement, a leg movement, a temperature (e.g., skin, core body, or other temperature), a galvanic skin response, or other parameter). In another example, a pattern associated with sleeping may comprise a set, or grouping, of sensory data corresponding to a movement or other characteristic or parameter associated with sleeping (e.g., temperature (e.g., skin, core body, or other temperature), a galvanic skin response, lying in a prone position for a period of time, lower heart rate, or other parameter). In still other examples, a pattern may be associated with other movements, motion, moods, states, activities, or events. If a given pattern is found in a pattern library, a control signal relating to the underlying activity or state may be generated and sent by a wearable device to a media application (e.g., an application that may be implemented using hardware, software, circuitry, or a combination thereof) that is configured to present media content (1708). Based on the control signal, a media file may be selected and presented (1710). For example, a given pattern may be recognized by band 1612 (FIG. 16A) as a shaking motion that is associated with playing a given list of music files (e.g., playlist). When the pattern is recognized and based on input provided by a user, band 1612 may be configured to send a control signal to skip to the next music file (e.g., song) in the playlist. As described in detail above in connection with FIG. 16A, any type of media file, content, or format may be used and is not limited to those described. Further, process 1700 and the above-described elements may be varied in order, function, detail, or other aspects, without limitation to examples provided.

FIG. 17B illustrates an exemplary process for device control using sensory input. Here, process 1720 begins by receiving an input from one or more sensors, which may be coupled to or in data communication with a wearable device (1722). Once received, the input is processed to determine a pattern (1724). Using the determined pattern, an operation is performed to reference a pattern library to determine whether a pre-defined or pre-existing control signal is identified (1726). If a control signal is found that correlates to the determined pattern, then wearable device 1612 (FIG. 16A) (e.g., data-capable strapband, or the like) may generate the identified control signal and send it to a given destination (e.g., another device or system in data communication with wearable device 1612). If, upon referencing a pattern library, a pre-defined or pre-existing control signal is not found, then another control signal may be generated and sent by wearable device 1612. Regardless, after determining a control signal to send using input from one or more sensors, wearable device 1612 generates the control signal for transmission to a device to either provide a device or device content control or management function (1728). In other examples, process 1720 and the above-described elements may be varied in order, function, detail, or other aspects, without limitation to examples provided.

FIG. 17C illustrates an exemplary process for wearable device data security. Here, process 1740 begins by receiving an input from one or more sensors, which may be coupled to or in data communication with a wearable device (1742). Once received, the input is processed to determine a pattern (1744). Using the determined pattern, an operation is performed to reference a pattern library to determine whether the pattern indicates a given signature that, for authentication purposes, may be used to perform or engage in a secure transaction (e.g., transferring funds or monies, sending or receiving sensitive personal information (e.g., social security numbers, account information, addresses, spouse/partner/children information, and the like)) (1746). Once identified, the signature may be transformed using various techniques (e.g., hash/hashing algorithms (e.g., MDA, SHA-1, and others, without limitation), checksum, encryption, encoding/decoding, and others, without limitation) into data formatted for transmission from wearable device 1612 (FIG. 16A) to another device and/or application (1748). After transforming the signature into data, the data is transmitted from wearable device 1612 to another device in data communication with the former (1750). In other examples, the data may be transmitted to other destinations, including intermediate networking routing equipment, servers, databases, data storage facilities, services, web services, and any other type of system or apparatus that is configured to authenticate the signature (i.e., transmitted data), without limitation. In still other examples, process 1740 and the above-described elements may be varied in order, function, detail, or other aspects, without limitation to examples provided.

FIG. 17D illustrates an exemplary process for movement languages in wearable devices. Here, process 1760 begins by receiving an input from one or more sensors, which may be coupled to or in data communication with a wearable device (1762). Once received, the input is processed to determine a pattern (1764). An inquiry may be performed to determine whether the pattern has been previously stored and, if not, it is stored as a new record in a database to indicate that a pattern is associated with a given set of movements, motions, activities, moods, states, or the like. If the determined pattern does have a previously stored pattern associated with the same or substantially similar set of sensory inputs (i.e., input received from one or more sensors), then the new pattern may be discarded or used to update the pre-defined or pre-existing pattern. In other examples, patterns that conflict with those previously stored may be evaluated differently to determine whether to store a given pattern in a pattern library. For example, if a pattern is identified as being associated with cycling, but is different from a previously stored pattern associated with cycling (e.g., on a different type of bicycle, on different terrain, using different gears, etc.), then the pattern may be stored as another (e.g., second, third, or other) cycling pattern. In another example, if a pattern is identified as being associated with sleeping, but is different from a previously stored pattern associated with sleeping, then the pattern may be stored as another sleeping pattern. In some examples, an algorithm may be implemented to determine whether a conflicting pattern should be stored as another version of a previously stored pattern, or discarded. In some examples, more than one pattern library may be stored on a wearable device. In some examples, a pattern library may be stored on a remote database and used by a wearable device that is in data communication with the remote database. After determining whether to store the pattern in a pattern library, the patterns may be aggregated in a movement library to develop a “movement language” (i.e., a collection of patterns) that may be used to interpret activities, states, or other user interactions with a wearable device in order to perform various functions, without limitation (1768). For example, once it is determined to store a pattern in a pattern library, the pattern may be added to a collection, or set, of patterns that are associated with an activity or motion (e.g., running, walking, swimming, jogging, jumping, shaking, turning, cycling, or others), a biological state (e.g., healthy, ill, diabetic, awake, asleep, or others), a physiological state (e.g., normal gait, limping, injured, sweating, high heart rate, high blood pressure, or others), or a psychological state (e.g., happy, depressed, angry, and the like). In other examples, process 1760 and the above-described elements may be varied in order, function, detail, or other aspects, without limitation to examples provided.

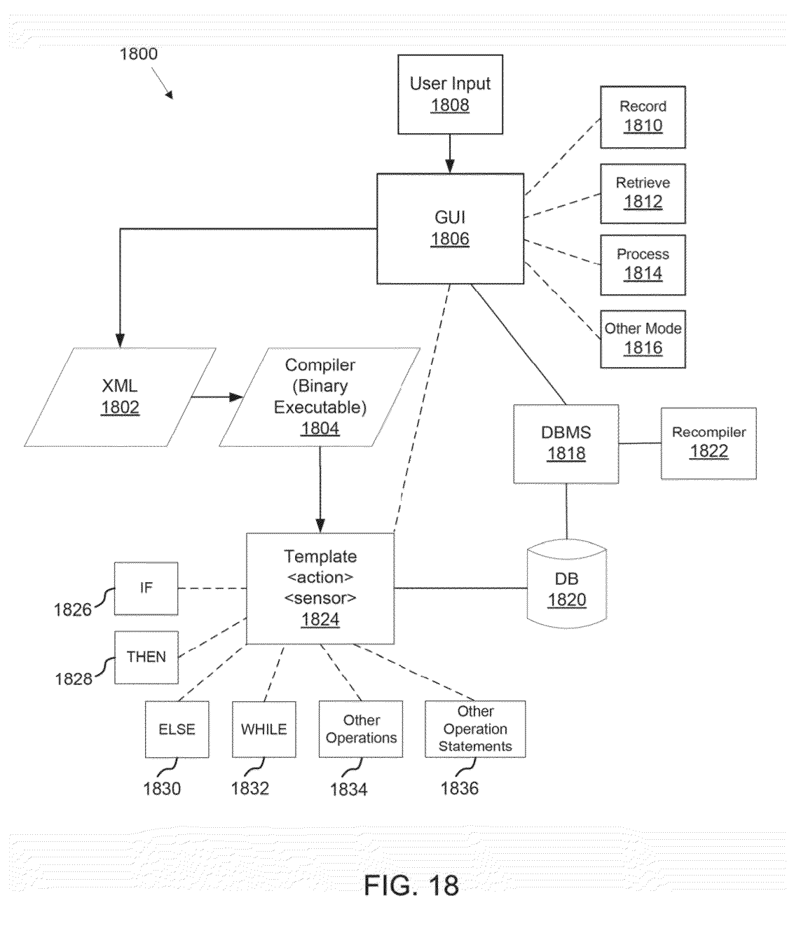

FIG. 18 illustrates an exemplary system for creating, storing, and performing other operations, with regard to motion profile templates. System 1800 may be configured to include XML 1802, compiler 1804, graphical user interface (GUI) 1806, user input 1808, modes 1810-1816, database management system (DBMS) 1818, database 1820, recompiler 1822, template 1824, and operations 1826-1836. In some examples, system 1800 may be implemented using XML 1802, which may be implemented using any type of XML markup language and may be compiled into binary form by compiler 1804 to form template 1824. In some examples, template 1824 may comprise tags denoting actions and sensors, for example associated with a motion profile (see, e.g., FIGS. 13 and 15). Template 1824 may include (i.e., support) simple operations, including IF operation 1826, THEN operation 1828, ELSE operation 1830, and WHILE operation 1832. Template 1824 also may include (i.e., support) other operations 1834 and other operation statements 1836. In some examples, system 1800 may be implemented with GUI 1806, which may be a user interface (i.e., graphical user interface) configured to enable a user to interact with (e.g., view output, provide input, or otherwise interact with) a system (i.e., system 1800). In some examples, GUI 1806 may receive user input 1808 in any format, including human-readable formats (e.g., by typing into a field, uploading data from another device, making selections on a form, or other human-readable input formats), which may be added or communicated to XML 1802 using GUI 1806. In some examples, GUI 1806 may be implemented with various modes of operation, including record mode 1810, retrieve mode 1812, process mode 1814 and other mode 1816. For example, record mode 1810 may enable a user to record a template. In some examples, user may record a template by performing an action using one or more data-capable bands and transmitting or uploading that data using GUI 1806. In another example, retrieve mode 1812 may enable a user to retrieve a template. For example, GUI 1806 may retrieve template 1824 from the database using DBMS 1818. In yet another example, process mode 1814 may enable a user to conduct other processes associated with a template (e.g., overwrite, download, etc.). In still another example, other mode 1816 may comprise yet an additional mode of operation available using GUI 1806. For example, other mode 1816 may comprise another manner in which a user may create a template by providing various types of user input 1808, as described above. In another example, GUI 1806 may be configured with a human-readable “drag-and-drop” interface that may enable a user to choose parameters for a template from various options or categories of options.

In some examples, template 1824 may be stored in database 1820. In some examples, template 1824 may be stored in binary form, and may be recompiled by recompiler 1822 (e.g., to display actions performed on template 1824 for a user, to be reviewed by a user, etc.). In some examples, template 1824 may describe an activity with biological, biometric, physical, physiological, psychological or other parameters. In other examples, one or more compiled templates may be formed into an applet (e.g., Java-based plug-in application, other Java applets, or other applets). In still other examples, template 1824 may be implemented with a priority for power management uses. In yet other examples, template 1824 may be sold, or bartered for, along with other templates on a marketplace (e.g., a fitness marketplace, Amazon Market Place™, eBay®, other online auction market, or other marketplace) or an SNS (e.g., Facebook®, Twitter®, etc.). Once created, template 1824 may be downloaded onto any data-capable band, including any of the data-capable bands described herein, either using GUI 1806 or other interfaces.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed is:

1. A method, comprising:
receiving motion-related data from a first sensor and user-related data from a second sensor over a time period, the first sensor and the second sensor being coupled to a wearable computing device;
determining a plurality of states of a user over the time period as a function of the motion-related data and the user-related data;
determining a trend as a function of the plurality of states of the user; and
displaying information associated with the trend at a user interface.

2. The method of claim 1, wherein the determining the plurality of states of the user over the time period is executed by a processor that is remote from the wearable computing device.

3. The method of claim 1, wherein the determining the plurality of states of the user over the time period is executed using an application that is received from a marketplace.

4. The method of claim 1, wherein the user-related data comprises galvanic skin response data.

5. The method of claim 1, wherein the user-related data comprises temperature data.

6. The method of claim 1, wherein the first sensor comprises an accelerometer.

7. The method of claim 1, further comprising:
storing the plurality of states of the user in a database.

8. The method of claim 1, further comprising:
storing the motion-related data and the user-related data in a database.

9. The method of claim 1, further comprising:
receiving environmental-related data from a third sensor that is coupled to the wearable computing device; and
determining the plurality of states of the user over the time period as a function of the motion-related data, the user-related data, and the environmental-related data.

10. The method of claim 1, wherein the displaying the information associated with the trend comprises:
displaying the information associated with the trend at a web browser.

11. A system, comprising:
a memory configured to store motion-related data received from a first sensor over a time period and user-related data received from a second sensor over the time period, the first sensor and the second sensor being coupled to a wearable computing device; and
a processor configured to determine a plurality of states of a user over the time period as a function of the motion-related data and the user-related data, to determine a trend as a function of the plurality of states of the user, and to display information associated with the trend at a user interface.

12. The system of claim 11, wherein the processor is located remotely from the wearable computing device.

13. The system of claim 11, wherein the processor is further configured to determine the plurality of states of the user using an application that is received from a marketplace.

14. The system of claim 11, wherein the user-related data comprises galvanic skin response data.

15. The system of claim 11, wherein the first sensor comprises an accelerometer.

16. The system of claim 11, wherein the processor is further configured to display the information associated with the trend as a graphic.

17. The system of claim 11, wherein the processor is further configured to store the plurality of states of the user in a database.

18. The system of claim 11, wherein the processor is further configured to store the motion-related data and the user-related data in a database.

19. The system of claim 11, wherein:
the memory is further configured to receive environmental-related data from a third sensor that is coupled to the wearable computing device; and
the processor is further configured to determine the plurality of states of the user over the time period as a function of the motion-related data, the user-related data, and the environmental-related data.

20. The system of claim 11, wherein the processor is further configured to display the information associated with the trend at a web browser.
